(19) United States
(12) Patent Application Publication
(10) Pub. No.: US 2003/0152610 A1
(43) Pub. Date: Aug. 14, 2003

(54) Cosmetic patch

(76) Inventors: David Rolf, Eden Prairie, MN (US); Teri Buseman, Minnetonka, MN (US); Dede Cooke, Wayzata, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/060,060

(22) Filed: Jan. 28, 2002

(51) Int. Cl. A61K 9/70

(52) U.S. Cl. 424/449; 442/123

(57) ABSTRACT

The present invention provides an adhesive patch. The adhesive patch includes a flexible backing having a front side and a back side and a cosmetic formulation positioned on at least a portion of the front side of the backing, in at least a portion of the front side of the backing, or on and in at least a portion of the front side of the backing. The cosmetic formulation includes a cosmetic agent; a solvent; a skin absorption enhancer; and at least one of a pressure sensitive adhesive and a polymer.

### BACKGROUND OF THE INVENTION

Cosmetic agents are known to improve the appearance of wrinkles, exfoliate the skin, hydrate the skin, and firm the skin. The formulations that include the cosmetic agents are typically sold as creams, lotions and/or gels. These formulations are often messy and inconvenient to use. For example, the user will typically wash their hands after using the product. Additionally, the user will typically be careful in not having the product come into contact with any clothes, jewelry, or any other surrounding objects (e.g., pillows, blankets, or bed sheets). These products will cause discomfort should they enter the eyes, mouth or nose of the user.

Adhesive patches are known to deliver pharmaceutical agents and cosmetic agents to the skin surfaces of humans. Many adhesive patches do not have an overall yield of product that is acceptable. These patches have a relatively high degree of penetration of the formulation in the backing. The adhesive patches typically have has a relatively low water content and a relatively high adhesiveness. Additionally, many of the adhesive patches do not effectively firm the skin surface of a mammal, improve the appearance of wrinkles in a mammal, effectively exfoliate the skin surface of the mammal, or effectively hydrate the skin surface of the mammal. When the adhesive skin patch is an adhesive face mask, it is typically a one piece face mask. These adhesive face masks are typically not adapted to fit a wide variety of shaped and sized faces.

As such, what is needed in the art is an adhesive skin patch useful to deliver an effective and known amount of cosmetic agent to the skin surface of a mammal (e.g., human). The adhesive patch will preferably be convenient, safe, and easy to use. Preferably, the adhesive patch will be safe for use near the eyes, mouth and nose. The adhesive patch will obviate the need to use messy and otherwise inconvenient gels, creams, or lotions. The adhesive patch will comply with FDA regulations. The adhesive patch will have an overall yield of product that is higher than current patches, will have an improved “holdout” of cosmetic formulation on the backing compared to current patches, and will have a lower degree of penetration of the cosmetic formulation in the backing of the device compared to known patches. The adhesive patch will have a higher water content than current patches. The adhesive patch will have less adhesiveness than current patches. The adhesive patch will effectively firm the skin surface of a mammal. The adhesive patch will effectively improve the appearance of wrinkles in a mammal. The adhesive patch will effectively exfoliate the skin surface of the mammal. The adhesive patch will effectively hydrate the skin surface of the mammal. The adhesive patch can be a two-piece face mask which compared to current one piece face masks, will not readily fold onto itself, and will be better adapted to fit a wider variety of shaped and sized faces.

### SUMMARY OF THE INVENTION

The present invention provides an adhesive skin patch useful to deliver an effective and known amount of cosmetic agent to the skin surface of a mammal (e.g., human). The adhesive patch is convenient, safe, and easy to use. Specifically, the adhesive patch is safe for use near the eyes, mouth and nose. The adhesive patch obviates the need to use messy and otherwise inconvenient gels, creams, or lotions. The adhesive patch complies with FDA regulations. The adhesive patch has an overall yield of product that is higher than current patches, has an improved “holdout” of cosmetic formulation on the backing compared to current patches, and has a lower degree of penetration of the cosmetic formulation in the backing of the device compared to known patches. The adhesive patch has a higher water content than current patches. The adhesive patch has less adhesiveness than current patches. The adhesive patch can effectively firm the skin surface of a mammal. The adhesive patch can effectively improve the appearance of wrinkles in a mammal. The adhesive patch can effectively exfoliate the skin surface of the mammal. The adhesive patch can effectively hydrate the skin surface of the mammal. The adhesive patch can be a two-piece face mask which compared to current one piece face masks, will not readily fold onto itself, and is better adapted to fit a wider variety of shaped and sized faces.

The present invention provides an adhesive patch. The adhesive patch includes a flexible backing having a front side and a back side and a cosmetic formulation positioned on at least a portion of the front side of the backing, in at least a portion of the front side of the backing, or on and in at least a portion of the front side of the backing. The cosmetic formulation includes a cosmetic agent; a solvent; a skin absorption enhancer; and at least one of a pressure sensitive adhesive and a polymer.

The present invention also provides another adhesive patch in the form of an adhesive mask. The adhesive patch includes a first portion and a second portion, each including a flexible backing having a front side and a back side and a cosmetic formulation positioned on at least a portion of the front side of the backing, in at least a portion of the front side of the backing, or on and in at least a portion of the front side of the backing. The cosmetic formulation includes a cosmetic agent, a solvent, a skin absorption enhancer and at least one of a pressure sensitive adhesive and a polymer. The first portion includes two apertures for the eyes of a person's face, such that the front side of the backing adhesively attaches to a skin surface of the person's face near the person's eyes. The second portion includes an aperture corresponding to the mouth of a person's face, such that the front side of the backing adhesively attaches to a skin surface of the person's face near the person's mouth.

The present invention also provides another adhesive patch in the form of an adhesive mask. The adhesive mask includes a first portion and a second portion, each including a flexible backing having a front side and a back side and a cosmetic formulation positioned on at least a portion of the front side of the backing, in at least a portion of the front side of the backing, or on and in at least a portion of the front side of the backing. The cosmetic formulation includes a cosmetic agent, a solvent, a skin absorption enhancer and at least one of a pressure sensitive adhesive and a polymer. The first portion is contoured to the nose, cheeks and eyes of a person's face and includes two apertures for the eyes of a person's face, such that the front side of the backing adhesively attaches to a skin surface of the person's face near the person's eyes. The second portion is contoured to the mouth and chin of a person's face and includes an aperture corresponding to the mouth of a person's face, such that the front side of the backing adhesively attaches to a skin surface of the person's face near the person's mouth.

The present invention also provides another adhesive patch in the form of an adhesive mask. The adhesive mask includes a flexible backing having a front side and a back side and a cosmetic formulation positioned on at least a portion of the front side of the backing, in at least a portion of the front side of the backing, or on and in at least a portion of the front side of the backing. The cosmetic formulation includes: a cosmetic agent selected from the group consisting of: an antioxidant, a collagen synthesis stimulator, a fibroblast growth stimulator, a collagen cross-linking inhibitor, caffeine, theophyline, and combinations thereof. The cosmetic formulation also includes a solvent; a skin absorption enhancer; and at least one of a pressure sensitive adhesive and a polymer.

The present invention also provides another adhesive patch in the form of an adhesive mask. The adhesive mask includes a flexible backing having a front side and a back side and a cosmetic formulation positioned on at least a portion of the front side of the backing, in at least a portion of the front side of the backing, or on and in at least a portion of the front side of the backing. The cosmetic formulation includes: a cosmetic agent selected from the group consisting of: Vitamin C, Vitamin E, Vitamin A, lactic acid, tartaric acid, citric acid, glycolic acid, malic acid, alpha-hydroxy octanoic acid, alpha-hydroxy caprylic acid, a mixed fruit acid, a sugar cane extract, salicylic acid, beta-hydroxybutanoic acid, tropic acid, trethocanic acid, a plant extract containing kinetin (6 furfurylaminopurine), Vitamin C, a copper containing peptide, retin A, a cytokine, a Fibroblast Growth Factor, aminoguanidine, carnosine, and combinations thereof. The cosmetic formulation also includes a solvent; a skin absorption enhancer; and at least one of a pressure sensitive adhesive and a polymer.

The present invention also provides another adhesive patch in the form of an adhesive mask. The adhesive mask includes a flexible backing having a front side and a back side and a cosmetic formulation positioned on at least a portion of the front side of the backing, in at least a portion of the front side of the backing, or on and in at least a portion of the front side of the backing. The cosmetic formulation includes: a cosmetic agent, a solvent, a skin absorption enhancer and at least one of a pressure sensitive adhesive and a polymer. The first portion includes two apertures for the eyes of a person's face, such that the front side of the backing adhesively attaches to a skin surface of the person's face near the person's eyes. The second portion includes an aperture corresponding to the mouth of a person's face, such that the front side of the backing adhesively attaches to a skin surface of the person's face near the person's mouth.

The present invention also provides another adhesive patch in the form of an adhesive mask. The adhesive mask includes a flexible backing having a front side and a back side and a cosmetic formulation positioned on at least a portion of the front side of the backing, in at least a portion of the front side of the backing, or on and in at least a portion of the front side of the backing. The cosmetic formulation includes: a cosmetic agent selected from the group consisting of: an antioxidant, a collagen synthesis stimulator, a fibroblast growth stimulator, a collagen cross-linking inhibitor, caffeine, theophyline, and combinations thereof;, a solvent, a skin absorption enhancer and at least one of a pressure sensitive adhesive and a polymer. The first portion includes two apertures for the eyes of a person's face, such that the front side of the backing adhesively attaches to a skin surface of the person's face near the person's eyes. The second portion includes an aperture corresponding to the mouth of a person's face, such that the front side of the backing adhesively attaches to a skin surface of the person's face near the person's mouth.

The present invention also provides another adhesive patch in the form of an adhesive mask. The adhesive mask includes a flexible backing having a front side and a back side and a cosmetic formulation positioned on at least a portion of the front side of the backing, in at least a portion of the front side of the backing, or on and in at least a portion of the front side of the backing. The cosmetic formulation includes: a cosmetic agent selected from the group consisting of: Vitamin C, Vitamin E, Vitamin A, lactic acid, tartaric acid, citric acid, glycolic acid, malic acid, alpha-hydroxy octanoic acid, alpha-hydroxy caprylic acid, a mixed fruit acid, a sugar cane extract, salicylic acid, beta-hydroxybutanoic acid, tropic acid, trethocanic acid, a plant extract containing kinetin (6 furfurylaminopurine), Vitamin C, a copper containing peptide, retin A, a cytokine, a Fibroblast Growth Factor, aminoguanidine, carnosine, and combinations thereof; a solvent; a skin absorption enhancer; and at least one of a pressure sensitive adhesive and a polymer. The first portion includes two apertures for the eyes of a person's face, such that the front side of the backing adhesively attaches to a skin surface of the person's face near the person's eyes. The second portion includes an aperture corresponding to the mouth of a person's face, such that the front side of the backing adhesively attaches to a skin surface of the person's face near the person's mouth.

The present invention also provides another adhesive patch in the form of an adhesive mask. The adhesive mask includes a flexible backing having a front side and a back side and a cosmetic formulation positioned on at least a portion of the front side of the backing, in at least a portion of the front side of the backing, or on and in at least a portion of the front side of the backing. The cosmetic formulation includes: a cosmetic agent, a solvent, a skin absorption enhancer and at least one of a pressure sensitive adhesive and a polymer. The first portion is contoured to the nose, cheeks and eyes of a person's face and includes two apertures for the eyes of a person's face, such that the front side of the backing adhesively attaches to a skin surface of the person's face near the person's eyes. The second portion is contoured to the mouth and chin of a person's face and includes an aperture corresponding to the mouth of a person's face, such that the front side of the backing adhesively attaches to a skin surface of the person's face near the person's mouth.

The present invention also provides a method for improving the appearance of wrinkles in a mammal in need thereof. The method includes applying to the surface of the skin of the mammal having the wrinkles or to the surface of the skin of the mammal at risk thereof, an adhesive patch of the present invention for a period of time effective to improve the appearance of the wrinkles.

The present invention also provides a method for exfoliating a skin surface of a mammal. The method includes applying to the skin surface of the mammal in need of such exfoliation an adhesive patch of the present invention for a period of time effective to adhere the adhesive skin patch to the skin surface, and removing the adhesive skin patch, thereby effectively exfoliating the skin surface.

The present invention also provides a method for hydrating a skin surface of a mammal. The method includes applying to the skin surface of the mammal in need of such hydration an adhesive patch of the present invention for a period of time effective to hydrate the skin surface.

The present invention also provides a method for firming a skin surface of a mammal. The method includes applying to the skin surface of the mammal in need of such firming an adhesive patch of any one of the present invention for a period of time effective to hydrate the skin surface.

### BRIEF DESCRIPTION OF THE FIGURES

FIG. 1 illustrates the front side of an adhesive patch of the present invention.

FIG. 2 illustrates the back side of an adhesive patch of the present invention.

FIG. 3 illustrates the front side of an adhesive patch of the present invention with a release liner attached to the patch.

FIG. 4 illustrates the back side of an adhesive patch of the present invention with a release liner attached to the patch.

FIG. 5 illustrates the back side of an adhesive patch of the present invention with a release liner attached to the patch, wherein the patch is partially detached from the release liner.

FIG. 6 illustrates the back side of an adhesive patch of the present invention wherein a portion of the patch is detached from the release liner.

FIG. 7 illustrates a top view of a specific patch of the present invention.

FIG. 8 illustrates a top view of a specific patch of the present invention.

FIG. 9 illustrates a specific adhesive skin patch of the present invention.

FIG. 10 illustrates an enlarged cross-sectional view of a specific patch of the present invention.

FIG. 11 illustrates a specific adhesive skin patch of the present invention.

FIG. 12 illustrates a specific adhesive skin patch of the present invention.

### DETAILED DESCRIPTION OF THE INVENTION

The present invention provides a unique adhesive vehicle. The vehicle has pressure sensitive adhesive qualities due to its composition and viscoelastic nature. The adhesive is hydrophilic and therefore water can dissolve into or evaporate from the adhesive, depending on the conditions to which it is exposed. This water exchange capability implies that if the adhesive is on a suitably porous backing and is applied to the skin, it will not be occlusive as most drug delivery patches are. The occlusive nature of conventional drug delivery patches is thought to play an important role in enhancing drug absorption, but also often results in greater incidence of skin irritation. The relatively low occlusiveness of the present invention can be envisioned to be a special adhesive ointment or gel which is water-breathable, such as a water washable or water soluble ointment or gel.

The present invention provides an ointment or gel on a backing. The ointment or gel includes an effective, known, and safe amount of a cosmetic agent that is useful in exfoliating skin. The backing is pliable and/or stretchable. Since the backing can be porous and/or vapor permeable, many consumers typically refer to the device as a “patch,” a “skin patch,” an “adhesive skin patch,” or a “face mask.” As such, the device (i.e., the ointment or gel on the backing) will herein be referred to as a patch, a skin patch, an adhesive skin patch and/or as a face mask. It is appreciated that those skilled in the art understand that the term “patch” is used to refer to the device and is not otherwise limiting in any manner.

The present invention provides an adhesive skin patch useful to deliver an effective and known amount of cosmetic agent to the skin surface of a mammal (e.g., human). The adhesive patch is convenient, safe, and easy to use. Specifically, the adhesive patch is safe for use near the eyes, mouth and nose. The adhesive patch obviates the need to use messy and otherwise inconvenient gels, creams, or lotions. The adhesive patch complies with FDA regulations. The adhesive patch has an overall yield of product that is higher than current patches, has an improved “holdout” of cosmetic formulation on the backing compared to current patches, and has a lower degree of penetration of the cosmetic formulation in the backing of the device compared to known patches. The adhesive patch has a higher water content than current patches. The adhesive patch has less adhesiveness than current patches. The adhesive patch can effectively firm the skin surface of a mammal. The adhesive patch can effectively improve the appearance of wrinkles in a mammal. The adhesive patch can effectively exfoliate the skin surface of the mammal. The adhesive patch can effectively hydrate the skin surface of the mammal. The adhesive patch can be a two-piece face mask which compared to current one piece face masks, will not readily fold onto itself, and is better adapted to fit a wider variety of shaped and sized faces.

As used herein, “holdout” refers to the physical properties of a backing, relating to the ability of a specific class of gels or ointments to penetrate, cross-link, wet, and/or cure within the matrix of the backing. A specific class of gels or ointments may or may not be able to penetrate a given backing. Upon penetration of a gel or ointment into a backing, the gel or ointment will cross-link, wet, or cure in the backing. As such, the holdout properties are a degree of the ability of the gel or ointment to affect the degree of penetration, cross-linking, wetting, and/or curing within the matrix of the backing. Those backings with superior holdout properties will typically prevent, decrease, or lessen the likelihood of the ointment or gel from wetting the backing; will typically increase the likelihood of the ointment or gel to cross-link within the matrix of the backing; will typically increase the likelihood of the ointment or gel to cure within the matrix of the backing; will typically increase the likelihood of the ointment or gel to partially penetrate the matrix of the backing. and/or will typically prevent, decrease, or diminish the likelihood of the ointment or gel to completely penetrate the matrix of the backing.

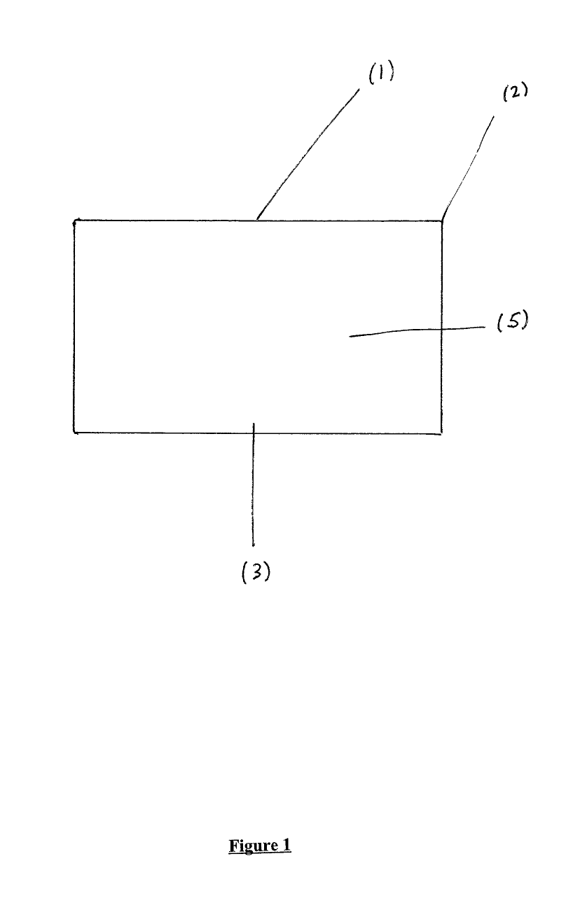

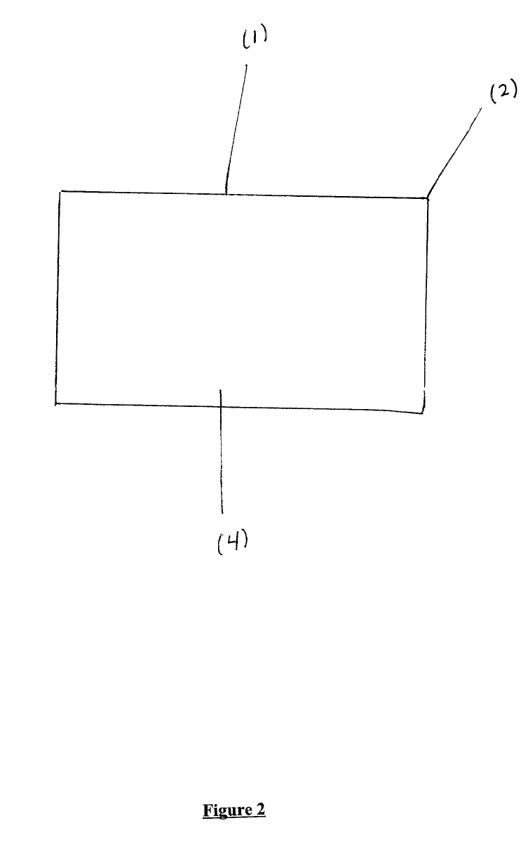

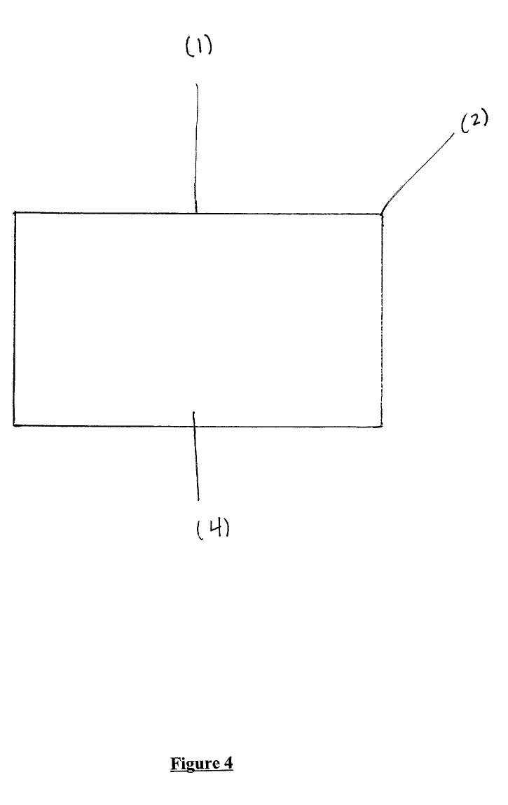

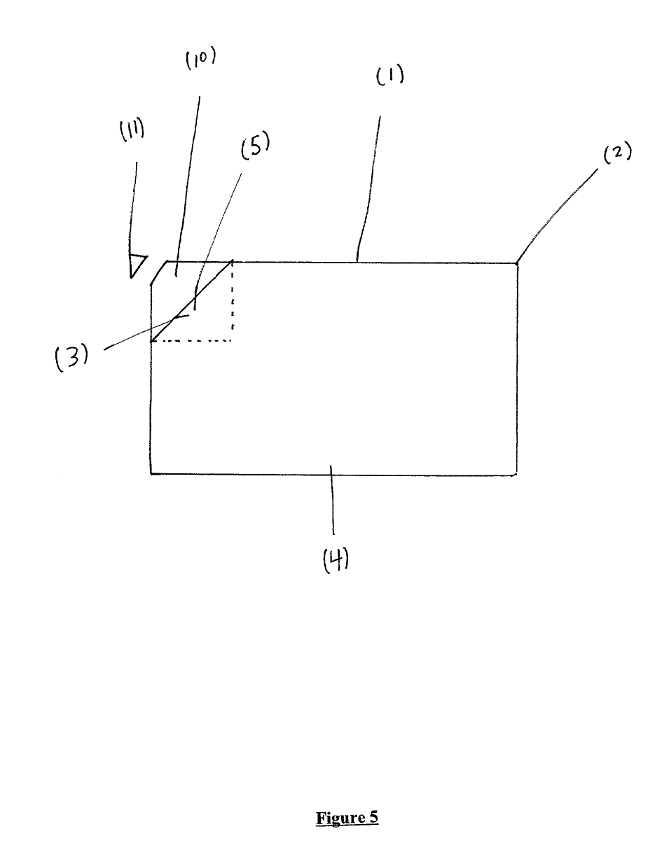

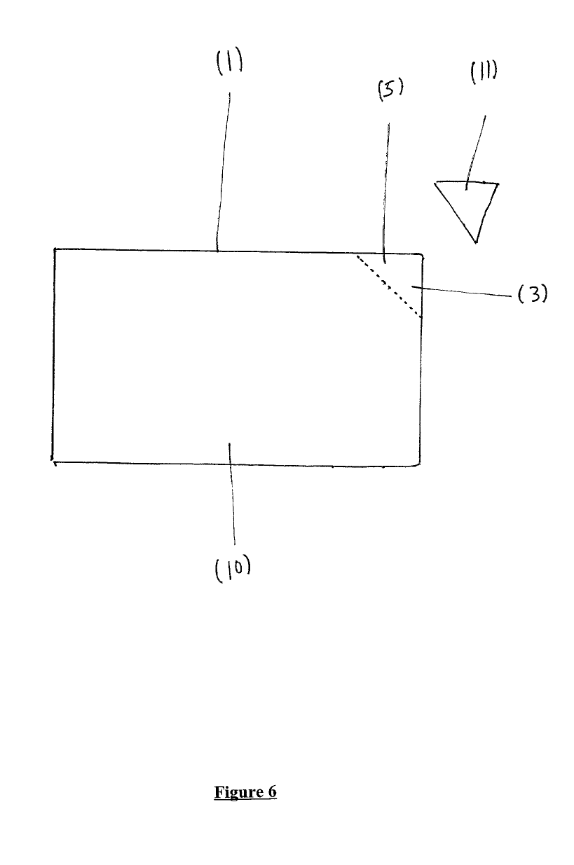

Referring to FIGS. 1-11, an adhesive patch 1 of the present invention is provided. The adhesive patch 1 includes a cosmetic formulation 5 located on at least a portion of the front side 3 of the backing 2, in at least a portion of the front side 3 of the backing 2, or on and in at least a portion of the front side 3 of the backing 2. Preferably, the cosmetic formulation 5 is partially embedded in at least a portion of the front side 3 of the backing 2. In addition to being located in at least a portion of the front side 3 of the backing 2, the cosmetic formulation 5 is located on a portion of the surface of front side 3 of the backing 2. Preferably, the cosmetic formulation 5 is located on the entire surface of the front side 3 of the backing 2.

Backing

The backing 2 is defined by a front side 3 (the side exposed to the skin during use) and a back side 4 (the side exposed to the environment during use). The backing 2 should be nonirritating to human skin. The backing 2 is a self-supporting sheet of water soluble or water insoluble, polymeric or natural material that provides strength and integrity for the cosmetic formulation 5. The backing 2 of the adhesive patch 1 can be vapor permeable. The backing 2 can also be porous, since porosity provides openings for receiving the cosmetic formulation 5 and it helps to assure that the adhesive skin patch 1 is vapor permeable. Specifically, the backing 2 can retain the cosmetic formulation 5 while allowing moisture from the skin to pass. The backing 2 can have any suitable thickness, provided the suitable thickness allows for a flexible, bendable, pliable, vapor permeable, and/or a stretchable sheet of water insoluble porous material. Specifically, the thickness of the backing 2 can be about 0.001 mm to about 5.0 mm, about 0.001 mm to about 3.0 mm, or about 0.025 mm to about 1.25 mm.

The backing 2 can be manufactured from any suitable material, provided the suitable material can form a flexible, bendable, pliable, and/or stretchable backing 2. The backing 2 includes a flexible porous sheet of water soluble or water insoluble material that provides support for the adhesive skin patch 1. The backing 2 can include water soluble or water insoluble polymeric fibers, a porous film, or any other kind of matrix with spaces within the matrix. A specific backing 2 is a lightweight, porous, pliable strip composed of a nonwoven fabric of polymeric or natural fibers such as polyester, cotton or cellulose fibers bonded together with a sizing resin. The backing 2 can be woven or nonwoven. Preferably, the backing 2 includes nonwoven fabric. Specifically, the backing 2 can include polyester, polyurethane, polyolefin, polyamide fibers, natural fibers, cotton fibers, polycellulose fibers, or any mixture thereof. Additional stable, water insoluble flexible sheet materials and methods for manufacturing the suitable backings 2 are disclosed, e.g., in U.S. Pat. No. 4,675,009; U.S. Pat. No. 5,536,263; U.S. Pat. No. 4,696,854; U.S. Pat. No. 5,741,510, and references cited therein, and are suitable as backings 2 according to the present invention. The infusion of the cosmetic formulation 5 into the backing 2 can be accomplished, e.g., with the use of a continuous process mixer, as disclosed, e.g., in U.S. Pat. No. 5,536,263, and references cited therein; or as discussed herein.

Alternatively, the backing 2 can be a non-woven backing 2 that is treated by coating: the front side 3 of the backing 2, the back side 4 of the backing 2, or both the front side 3 and back side 4 of the backing 2; with a silicone-containing compound. Suitable silicone-containing compounds include, e.g., polydimethyl siloxanes, dialkylsiloxanes, dimethylsiloxo vinyl alkenes, dialkylsiloxo vinyl alkenes, dimethylsiloxo acrylates, dialkylsiloxo acrylates, vinyl terminated polydimethylsiloxane, and vinyl terminated polydialkylsiloxane. The exemplary silicone-containing compounds are commercially available from, e.g., Goldschmidt Chemical Corp. (Essen, Germany); GE Silicones (Waterford, N.Y.); Wacker Silicone Corp. (Adrian, Mich.); and Dow Coming Corp. (Midland, Mich.).

The backing 2 can be manufactured from a suitable non-woven fabric that is commercially available from, e.g., Freudenberg Faservliesstoffe KG (Weinham, Germany); Sontara Technologies (division of DuPont Corporation) (Old Hickory, Tenn.); Lystil S. A. (Brignoud Cedex, France); Dexter Nonwovens (Windsor Locks, Conn.); and Chicopee (New Brusnwick, N.J.). Other commercial vendors that supply suitable non-woven fabrics can be found at the Technical Textile website (http://www.technical-textiles.net/technical-textiles-index/orgL.htm).

It has surprisingly been discovered that the use of a treated backing, such as a fluorocarbon treated non-woven backing, typically increases the yield of an adhesive patch. The use of a backing material that has been treated with a sizing agent allows for the effective control of the rate of penetration, such that the gel or ointment has solidified after it has begun to penetrate the backing, but before it has passed completely through the backing. In addition, the use of a backing material that has been treated with a sizing agent allows for the effective control of the depth to which the ointment or gel will easily penetrate before solidifying. It has surprisingly been discovered that increasing the control of the rate at which the ointment or gel penetrates the backing typically improves the overall yield of the production process by reducing the amount of material which must be discarded because the back side of the backing has become too tacky for either processing or for consumer acceptance.

At least a portion of the backing 2 can be treated with a sizing agent 8 such that the portion of the backing 2 that is treated with the sizing agent 8 has a surface energy of about 20 dynes/cm² to about 65 dynes/cm². Specifically, the portion of the backing 2 that is treated with the sizing agent 8 can have a surface energy of about 27 dynes/cm² to about 56 dynes/cm². The sizing agent 8 lowers the surface energy of the portion of the backing 2 that is treated with the sizing agent 8. Any suitable sizing agent 8 can be employed, provided the portion of the backing 2 that is treated with the sizing agent 8 has a surface energy of about 20 dynes/cm² to about 65 dynes/cm². Suitable sizing agents 8 include, e.g., fluorocarbon solutions, silicone-containing compounds, and combinations thereof. Specifically, the backing 2 can be a non-woven backing 2 that is treated with a fluorocarbon. For example, the fluorocarbon treated backing 2 can be, e.g., Vilmed M1585 W/HY, Vilmed M1585H/HY, Vilmed M1586 W/HY, Vilmed M1586 H/HY, Vilmed M1570, Vilmed M1573 F, Vilmed M1573 FH, Vilmed M1577 F, Vilmed M1578 F, or Vilmed M1578 FH; which are all commercially available from Freudenberg Faservliesstoffe KG (Weinham, Germany). Alternatively, the silicone treated backing 2 can be a non-woven backing 2 that is coated with one or more silicone-containing compounds, e.g., a polydimethyl siloxane, a dialkylsiloxane, a dimethylsiloxo vinyl alkene, a dialkylsiloxo vinyl alkenes, a dimethylsiloxo acrylate, a dialkylsiloxo acrylate, a vinyl terminated polydimethylsiloxane, and a vinyl terminated polydialkylsiloxane.

At least a portion of the backing 2 can be treated with the sizing agent 8. The portion of the backing 2 that is treated with the sizing agent 8 can be that portion of the backing 2 that can typically include the cosmetic formulation 5. The entire surface of the front side 3 of the backing 2 can be treated with the sizing agent 8 or a portion of the surface of the front side 3 of the backing 2 can be treated with the sizing agent 8. Preferably, the entire surface of the front side 3 of the backing 2 can be treated with the sizing agent 8. In addition to the surface of the front side 3 of the backing 2 being treated with the sizing agent 8, the sizing agent 8 can penetrate at least a portion of the underlying surface (e.g., one-tenth to about nine-tenths the thickness, or about one-fourth to about nine-tenths the thickness) of the backing 2. Specifically, the sizing agent 8 can penetrate the entire underlying surface of the backing 2.

Suitable fluorocarbon treated backings 2 include, e.g., Vilmed M1585 W/HY, Vilmed M1585H/HY, Vilmed M1586 W/HY, Vilmed M1586 H/HY, Vilmed M1570, Vilmed M1573 F, Vilmed M1573 FH, Vilmed M1577 F, Vilmed M1578 F, and Vilmed M1578 FH; which are all commercially available from Freudenberg Faservliesstoffe KG (Weinham, Germany).

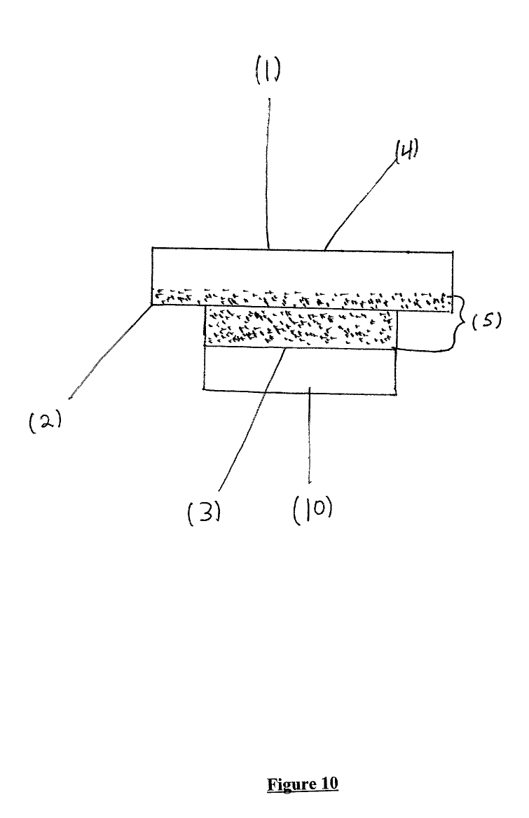

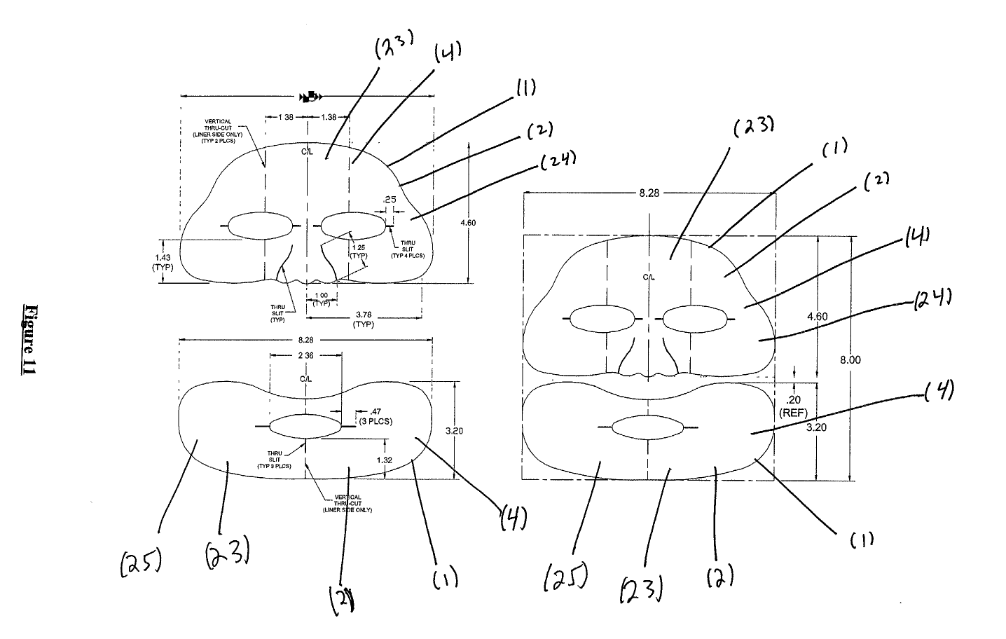

As shown in FIGS. 1-11, the backing 2 includes a front side 3 and a back side 4. The adhesive skin patch 1 includes a cosmetic formulation 5 located in at least a portion of the front side 3 of the backing 2, on at least a portion of the front side 3 of the backing 2, or on and in at least a portion of the front side 3 of the backing 2. As such, the cosmetic formulation 5 can be located on the entire surface of the front side 3 of the backing 2 or the cosmetic formulation 5 can be located on a portion of the surface of the front side 3 of the backing 2. Preferably, the cosmetic agent 5 can be located on the entire surface of the front side 3 of the backing 2. In addition to being located on the surface of the front side 3 of the backing 2, the cosmetic agent 5 can be located in at least a portion of the underlying surface of the front side 3 of the backing 2 (i.e., the cosmetic formulation 5 can be partially embedded into the backing 2). As shown in FIG. 10, the cosmetic formulation 5 can penetrate a substantial portion of the front side 3 of the backing 2, as disclosed, e.g., in U.S. Pat. No. 5,536,263, and references cited therein. For example, the cosmetic formulation 5 can penetrate about one-tenth to about nine-tenths the thickness of the backing 2, or about one-fourth to about nine-tenths the thickness of the backing 2. As such, the cosmetic formulation 5 can be partially embedded into the backing 2. Preferably, the cosmetic agent 5 can be located on the entire front side 3 of the backing 2 and partially in the front side 3 of the backing 2 (i.e., the cosmetic formulation 5 is partially embedded into the backing 2). Alternatively, a portion of the front side 3 of the backing 2 can include the cosmetic formulation 5 and other portions of the front side 3 of the backing 2 can include any combination of the pressure sensitive adhesive 14, cosmetic agent 15, and solvent 13. For example, a central circular portion of the front side 3 of the backing 2 can include the cosmetic formulation 5 and solvent 13 while the remaining portions of the front side 3 of the backing 2 include only the pressure sensitive adhesive 14. The cosmetic formulation 5, when partially embedded into the front side 3 of the backing 2, imparts strength and structure into the adhesive patch 1. For example, when the cosmetic formulation 5 is partially embedded into the backing 2, the likelihood that the adhesive patch 1 will tear apart when separated from the release liner 10 or when removed from the skin after use, is minimized. When the adhesive skin patch 1 is placed upon the skin of a patient (e.g., human), the cosmetic formulation 5 can be in continuous contact with the skin surface of the patient.

Preferably, the adhesive skin patch 1, upon contact with skin, will allow the skin to breathe. More preferably, the adhesive skin patch 1, upon prolonged contact with skin, will hold in place the cosmetic formulation 5 and will permit the skin to breathe over prolonged periods of time typically experienced with the use of the patch, e.g., up to about 24 hours, up to about 12 hours, up to about 8 hours, or up to about 6 hours.

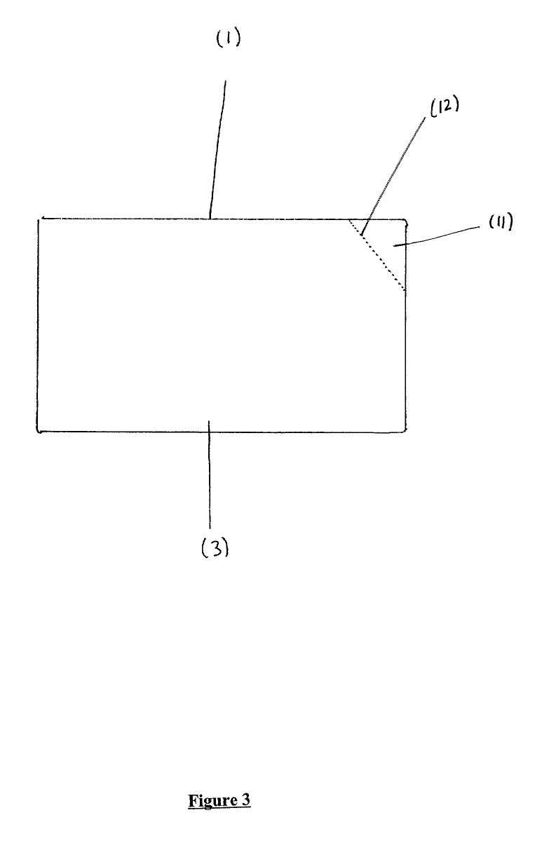

As shown in FIGS. 3, 5, 6, and 10, the adhesive skin patch 1 can be reversibly attached to a release liner 10. The release liner 10 helps to maintain the adhesiveness of the adhesive skin patch 1 prior to use, such as during manufacturing, packaging, shipping, and/or storage. Any suitable release liner 10 can be employed for use in the present invention. Suitable release liners 10 are readily known to those of skill in the art. See, e.g., U.S. Pat. No. 4,675,009; U.S. Pat. No. 5,536,263; U.S. Pat. No. 4,696,854; U.S. Pat. No. 5,741,510, and references cited therein for further descriptions of release liners 10 useful in the present invention. The release liner 10 can include a perforation 12 that allows the tab section 11 of the release liner 10 to be removed (see, FIGS. 3, 5, and 6). Removal of the tab section 11 of the release liner 10 allows the adhesive skin patch 1 to be removed from the release liner 10 with relative ease.

Cosmetic Agent

As used herein, a “cosmetic agent” refers to a substance suitable for topical administration (e.g., skin, hair, and/or nails) for purposes of beatifying a mammal (e.g., human) in accordance with cultural dictates. See, e.g., Stedman's Medical Dictionary, 25^{th} edition, illustrated, p.362 (1991).

Any suitable cosmetic agent 15 can be employed, provided the cosmetic agent 15 effectively improves the appearance of the skin surface of a mammal (e.g., human). Improving the skin surface of the mammal (e.g., human) can include, e.g., any one or more of the following: (1) diminishing the presence of, preventing, improving the appearance of and/or treating wrinkles and/or fine lines present on a skin surface; (2) exfoliating the skin surface; (3) firming the skin surface; and (4) hydrating the skin surface.

As used herein, “exfoliate” refers to the removal or detachment of superficial cells of an epithelium surface or horny layer (the stratum corneum) of the epidermis. Preferably, the skin cells, upon removal, are dead and are from the outermost one or two layers of the stratum corneum. The cosmetic formulation 5 possesses suitable physical properties (e.g., sufficient adhesiveness) to effectively remove or detach superficial cells of the epithelium surface or stratum corneum of the epidermis. The adhesive patch 1 can be applied to the skin surface to be exfoliated for an effective period of time, e.g., from about 1 second to about 24 hours, from about 1 minute to about 12 hours, from about 10 minutes to about 8 hours. After such effective period of time, the adhesive patch 1 can be removed from the skin surface. Such exfoliation of the skin is believed to (1) diminish the presence of, prevent, improve the appearance of and/or treat wrinkles and/or fine lines present on a skin surface; and (2) firm the skin surface.

Preferably, the cosmetic agent 15 will be safe for topical administration over prolonged periods of time (e.g., up to about 1 hour, up to about 4 hours, up to about 8 hours, up to about 12 hours, or up to about 24 hours). The cosmetic agent 15 will preferably remain stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1.

Suitable cosmetic agents 15 include, e.g., antioxidants, collagen synthesis stimulators, fibroblast growth stimulators, collagen cross-linking inhibitors, caffeine, theophyline, and combinations thereof.

Suitable antioxidants include, e.g., free radical scavengers (e.g., alpha hydroxy acids, beta hydroxy acids, Vitamin C, Vitamin E, Vitamin A, lycopene, tumeric, green tea, white tea, acceptable salts thereof (e.g., ammonium salts), acceptable derivatives thereof, and combinations thereof.

Suitable alpha hydroxy acids include, e.g., lactic acid, tartaric acid, citric acid, glycolic acid, malic acid, alpha-hydroxy octanoic acid, alpha-hydroxy caprylic acid, mixed fruit acids, sugar cane extracts, and acceptable salts (e.g., ammonium salts) thereof.

Suitable beta hydroxy acid include, e.g., salicylic acid, beta-hydroxybutanoic acid, tropic acid, trethocanic acid, and acceptable salts (e.g., ammonium salts) thereof.

Suitable collagen synthesis stimulator include, e.g., plant extracts containing kinetin (6 furfurylaminopurine), Vitamin C, copper containing peptides, and combinations thereof.

Suitable fibroblast growth stimulators include, e.g., copper containing peptides, retin A, cytokines, and combinations thereof.

Suitable cytokines include, e.g., Fibroblast Growth Factors.

Suitable collagen cross-linking inhibitors include, e.g., aminoguanidine, carnosine, or a combination thereof.

Specifically, the cosmetic agent 15 can include tourmaline, Vitamin C, Vitamin E, Vitamin A, lactic acid, tartaric acid, citric acid, glycolic acid, malic acid, alpha-hydroxy octanoic acid, alpha-hydroxy caprylic acid, a mixed fruit acid, a sugar cane extract, salicylic acid, beta-hydroxybutanoic acid, tropic acid, trethocanic acid, a plant extract containing kinetin (6 furfurylaminopurine), Vitamin C, a copper containing peptide, retin A, a cytokine, a Fibroblast Growth Factor, aminoguanidine, carnosine, or a combination thereof.

The cosmetic agent 15 can be present in any appropriate and suitable amount. Specifically, the cosmetic agent 15 can be present in about 0.01 wt. % to about 99.9 wt. % of the cosmetic formulation 5. Typically, the amount of cosmetic agent 15 present in the cosmetic formulation 5 will depend upon the specific compound or compounds employed as the cosmetic agent 15. Preferably, the amount of cosmetic agent 15 will comply with any relevant FDA regulations.

The amount of cosmetic agent 15 present in the cosmetic formulation 5 will typically depend upon the specific compound or compounds employed as the cosmetic agent 15. The cosmetic agent 15 can typically be present up to about 99.9 wt. % of the cosmetic formulation 5, up to about 10.0 wt. % of the cosmetic formulation 5, up to 4.0 wt. % of the cosmetic formulation 5, up to about 2.5.0 wt. % of the cosmetic formulation 5, or up to about 1.0 wt. % of the cosmetic formulation 5. Specifically, the cosmetic agent 15 can be present in about 0.01 wt. % of the cosmetic formulation 5, in about 0.05 wt. % to about 1.0 wt. % of the cosmetic formulation 5, or in about 0.1 wt. % to about 1.0 wt. % of the cosmetic formulation 5.

The adhesive skin patch 1 includes a cosmetic agent 15 located in at least a portion of the front side 3 of the backing 2, on at least a portion of the front side 3 of the backing 2, or on and in at least a portion of the front side 3 of the backing 2. As such, the cosmetic agent 15 can be located on the entire surface of the front side 3 of the backing 2 or the cosmetic agent 15 can be located on a portion of the surface of the front side 3 of the backing 2. Preferably, the cosmetic agent 15 can be located on the entire surface of the front side 3 of the backing 2.

In addition to being located on the surface of the front side 3 of the backing 2, the cosmetic agent 15 can be located in at least a portion of the underlying surface of the front side 3 of the backing 2 (i.e., the cosmetic agent 15 can be partially embedded into the backing 2). As shown in FIG. 10, the cosmetic agent 15 can penetrate a substantial portion of the front side 3 of the backing 2, as disclosed, e.g., in U.S. Pat. No. 5,536,263, and references cited therein. For example, the cosmetic agent 15 can penetrate about one-tenth to about nine-tenths the thickness of the backing 2, or about one-fourth to about nine-tenths the thickness of the backing 2. As such, the cosmetic agent 15 can be partially embedded into the backing 2.

Preferably, the cosmetic agent 15 can be located on the entire front side 3 of the backing 2 and partially in the front side 3 of the backing 2 (i.e., the cosmetic agent 15 is partially embedded into the backing 2). Alternatively, a portion of the front side 3 of the backing 2 can include the cosmetic agent 15 and other portions of the front side 3 of the backing 2 can include any combination of the pressure sensitive adhesive 14 and solvent 13. For example, a central circular portion of the front side 3 of the backing 2 can include the cosmetic agent 15 while the remaining portions of the front side 3 of the backing 2 include only the pressure sensitive adhesive 14 and solvent 13. When the adhesive skin patch 1 is placed upon the skin of a patient (e.g., human), the cosmetic agent 15 can be in continuous contact with the skin surface of the patient.

Solvent

The solvent 13 can act as a carrier for, and preferably can dissolve or suspend the cosmetic agent 15, pressure sensitive adhesive 14, and/or polymer 9. Some cosmetic agents 15 will be at least partially soluble in the chosen solvent 13. In such an embodiment, the solvent 13 dissolves the cosmetic agents 15. Other cosmetic agents 15, however, will not readily dissolve in the solvent 13 (i.e., the cosmetic agent 15 is not soluble in the solvent 13 to any appreciable degree). In such an instance, the solvent 13 can effectively suspend the cosmetic agents 15 while dissolving the pressure sensitive adhesive 14, and/or polymer 9.

Any suitable solvent 13 can be employed, provided the solvent 13 effectively dissolves or suspends the cosmetic agent 15, pressure sensitive adhesive 14, and/or polymer 9; and the solvent 13 remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1.

The solvent 13 can include one or more organic compounds, one or more inorganic compounds, or mixtures thereof. Preferably, the solvent 13 will include one or more organic compounds, e.g., esters, terpenes, alcohols, ketones, aldehydes, fatty acids, partially or fully esterified fatty acids, wherein the structures are cyclic, non cylcic (e.g., alkyl), alicyclic (i.e., a bridged ring compound), or aromatic, as well as organic compounds having combinations of these functional groups. Suitable exemplary solvents 13 are disclosed, e.g., in Aldrich Handbook of Fine Chemicals, 2000-2001 (Milwaukee, Wis.).

Preferably, the solvent 13 includes a polyhydric alcohol, water, or a combination thereof. The polyhydric alcohol can be propylene glycol, ethylene glycol, triethylene glycol, or a combination thereof. Additional suitable solvents 13 include, e.g., glycerin; triacetin; diethylene glycol ethyl ether; diethylene glycol ethyl ether acetate; 1,3-propane diol; 2-methyl-1,3-propane diol; glycerol ricinoleate; PEG-6 caprylic/capric glycerides; caprylic/capric triglycerides; propyleneglycol dicaprylate/dicaprate; glycerol monostearate; glycerol monocaprylate; glycerol monolaurate; neopentyl alcohol; 1-hexadecanol; hydroxypropyl beta-cyclodextrin; vitamin E; vitamin E acetate; deoxycholic acid; taurodeoxycholic acid; 3-[(3-cholamidopropyl)dimethylammonio]-1-propane-sulfonate; BigCHAP; cholic acid; cholesterol NF; propylene carbonate; lecithin; a pharmaceutically acceptable salt thereof; or a combination thereof.

The solvent 13 can be employed in any suitable amount, provided the amount of solvent 13 is effective to dissolve the cosmetic agent 15, pressure sensitive pressure sensitive adhesive 14, and/or polymer 9; and the effective amount of solvent 13 remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Specifically, the solvent 13 can be present in about 1.0 wt % to about 70.0 wt. %; in about 3.0 wt % to about 50.0 wt. %; or in about 5 wt. % to about 30 wt. % of the cosmetic formulation 5. Typically, the amount of solvent 13 will depend on the compound or compounds employed as the solvent 13. For example, a polyhydric alcohol can be present up to about 70 wt. % of the cosmetic formulation 5; or in about 20.0 wt. % to about 60.0 wt. % of the cosmetic formulation 5; and water can be present in about 2.0 wt. % to about 50.0 wt. % of the cosmetic formulation 5.

The solvent 13 can be located in at least a portion of the front side 3 of the backing 2, on at least a portion of the front side 3 of the backing 2, or on and in at least a portion of the front side 3 of the backing 2. As such, the solvent 13 can be located on the entire surface of the front side 3 of the backing 2 or the solvent 13 can be located on a portion of the surface of the front side 3 of the backing 2. Preferably, the solvent 13 can be located on the entire surface of the front side 3 of the backing 2. In addition to being located on the surface of the front side 3 of the backing 2, the solvent 13 can be located in at least a portion of the underlying surface of the front side 3 of the backing 2 (i.e., the solvent 13 can be partially embedded into the backing 2).

As shown in FIG. 10, the solvent 13 can penetrate a substantial portion of the front side 3 of the backing 2, as disclosed, e.g., in U.S. Pat. No. 5,536,263, and references cited therein. For example, the solvent 13 can penetrate about one-tenth to about nine-tenths the thickness of the backing 2, or about one-fourth to about nine-tenths the thickness of the backing 2. As such, the solvent 13 can be partially embedded into the backing 2. Preferably, the solvent 13 can be located on the entire front side 3 of the backing 2 and partially in the front side 3 of the backing 2 (i.e., the solvent 13 is partially embedded into the backing 2). Alternatively, a portion of the front side 3 of the backing 2 can include the solvent 13 and other portions of the front side 3 of the backing 2 can include any combination of the pressure sensitive adhesive 14 and cosmetic agent 15. When the adhesive skin patch 1 is placed upon the skin of a patient (e.g., human), the solvent 13 can be in continuous contact with the skin surface of the patient.

Pressure Sensitive Adhesive (P.S.A.)

Any suitable pressure sensitive pressure sensitive adhesive 14 can be employed, provided the pressure sensitive pressure sensitive adhesive 14 provides the requisite adhesiveness to the adhesive skin patch 1 and the pressure sensitive adhesive 14 remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. It is appreciated that the suitable pressure sensitive adhesives 14 are known to those skilled in the art. Suitable pressure sensitive adhesives 14 are disclosed, e.g., in U.S. Pat. No. 4,675,009; U.S. Pat. No. 5,536,263; U.S. Pat. No. 4,696,854; U.S. Pat. No. 5,741,510, and references cited therein. Preferably the pressure sensitive adhesive 14 is an acrylic ester copolymer.

Any suitable amount of pressure sensitive adhesive 14 can be employed, provided the amount of pressure sensitive adhesive 14 effectively provides the requisite adhesiveness to the adhesive skin patch 1 and the effective amount of the pressure sensitive adhesive 14 remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. The cosmetic formulation 5 can include a pressure sensitive adhesive 14 up to about 50 wt. %, up to about 10.0 wt. %, or up to about 15.0 wt. % of the cosmetic formulation 5. Typically, the suitable amount of pressure sensitive adhesive 14 will depend upon the specific pressure sensitive adhesive 14 employed. For example, the pressure sensitive adhesive 14 can include one or more acrylic ester copolymers. Each of the one or more acrylic ester copolymers can be present up to about 20.0 wt. % of the cosmetic formulation 5. Specifically, each of the acrylic ester copolymers can be present up to about 40.0 wt. % of the cosmetic formulation 5, or up to about 30.0 wt. % of the cosmetic formulation 5. More specifically, all of the one or more acrylic ester copolymers, when combined, can be present in about 3.0 wt. % to about 40.0 wt. % of the cosmetic formulation 5, or in about 5.0 wt. % to about 30.0 wt. % of the cosmetic formulation 5. As such, the total amount of acrylic ester copolymers can be about 3.0 wt. % to about 40.0 wt. % of the cosmetic formulation 5, or about 5.0 wt. % to about 30.0 wt. % of the cosmetic formulation 5.

Alternatively, the pressure sensitive adhesive 14 can include a hot melt pressure sensitive adhesive 14 or solvent based pressure sensitive adhesive 14 (e.g., polyacrylate, polyisobutylene, and polybutene), rubber, silicone based pressure sensitive adhesives 14 (e.g., polydimethylsiloxane and resin mixtures), polystyrene-polybutadiene-polystyrene, polystyrene-polyisoprene-polystyrene, polystyrene-poly(ethylene-butylene)-polystyrene block polymers, or any combination thereof. In addition, the adhesive 14 can include a resin emulsion adhesive, wherein the resin emulsion adhesive can include vinyl acetate resin, acrylic ester copolymer, vinyl acetate/diocyl maleate copolymer, acrylic copolymer, or any combination thereof.

Other suitable pressure sensitive adhesives 14 are disclosed, e.g., in U.S. Pat. No. 4,675,009; U.S. Pat. No. 5,536,263; U.S. Pat. No. 4,696,854; U.S. Pat. No. 5,741,510, and references cited therein.

The pressure sensitive adhesive 14 can be located in at least a portion of the front side 3 of the backing 2, on at least a portion of the front side 3 of the backing 2, or on and in at least a portion of the front side 3 of the backing 2. As such, the pressure sensitive adhesive 14 can be located on the entire surface of the front side 3 of the backing 2 or the pressure sensitive adhesive 14 can be located on a portion of the surface of the front side 3 of the backing 2. Preferably, the pressure sensitive adhesive 14 can be located on the entire surface of the front side 3 of the backing 2. In addition to being located on the surface of the front side 3 of the backing 2, the pressure sensitive adhesive 14 can be located in at least a portion of the underlying surface of the front side 3 of the backing 2 (i.e., the pressure sensitive adhesive 14 can be partially embedded into the backing 2). As shown in FIG. 10, the pressure sensitive adhesive 14 can penetrate a substantial portion of the front side 3 of the backing 2, as disclosed, e.g., in U.S. Pat. No. 5,536,263, and references cited therein. For example, the pressure sensitive adhesive 14 can penetrate about one-tenth to about nine-tenths the thickness of the backing 2, or about one-fourth to about nine-tenths the thickness of the backing 2. As such, the pressure sensitive adhesive 14 can be partially embedded into the backing 2. Preferably, the pressure sensitive adhesive 14 can be located on the entire front side 3 of the backing 2 and partially in the front side 3 of the backing 2 (i.e., the pressure sensitive adhesive 14 is partially embedded into the backing 2). Alternatively, a portion of the front side 3 of the backing 2 can include the pressure sensitive adhesive 14 and other portions of the front side 3 of the backing 2 can include any combination of the solvent 13 and cosmetic agent 15. The pressure sensitive adhesive 14, being partially embedded into the front side 3 of the backing 2, imparts strength and structure into the adhesive patch 1. For example, when the pressure sensitive adhesive 14 is partially embedded into the backing 2, the likelihood that the adhesive patch 1 will tear apart when separated from the release liner 10 or when removed from the skin after use, is minimized. When the adhesive skin patch 1 is placed upon the skin of a patient (e.g., human), the pressure sensitive adhesives 14 can be in continuous contact with the skin surface of the patient.

Polymer

The pressure sensitive adhesive 14 can include one or more polymers 9. The polymer 9 provides structure and strength to the pressure sensitive adhesive 14, provides structure and strength to the cosmetic formulation 5, and/or provides adhesiveness to the adhesive skin patch 1. Any suitable polymer 9 can be employed, provided the polymer 9 provides structure and strength to the pressure sensitive adhesive 14, provides structure and strength to the cosmetic formulation 5, and/or provides adhesiveness to the adhesive skin patch 1; and the polymer 9 remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1.

Suitable polymers 9 include natural polymers and synthetic polymers. Specifically, the polymer 9 can include, e.g., a polymer that includes a quaternary ammonium salt, gum tragacanth, gum Ghatti, gum agar, pectin, chitin, a derivative of chitin, carrageenan, a calcium cross-linked alginate, a cross-linked polymer by means of boron or a di- or tri-valent metal ion, an aldehyde cross-linked gelatin, gelatin, karaya, a polyacrylamide, a polyacrylic acid, xanthan gum, guar gum, a natural polymer, a synthetic polymer, a hydrophilic polymer, a hydrocolloidal polymer, starch, a starch derivative or graft copolymer, vinyl acetate copolymer, polyvinyl pyrrolidone, polyethylene oxide, algin, derivatives of algin, a polyacrylate, polymaleic acid, polymaleic anhydride, a polyurethane, a polyurea, gum acacia, locust bean gum, modified guar gum, maltodextrin, carboxymethyl cellulose, carboxypropyl cellulose, polyvinyl alcohol, poly AMPS, sodium polyacrylate, or a mixture thereof. Other suitable polymers 9 are disclosed, e.g., in U.S. Pat. No. 4,675,009; U.S. Pat. No. 5,536,263; U.S. Pat. No. 4,696,854; U.S. Pat. No. 5,741,510, and references cited therein. Preferably, the polymer 9 can include polyacrylamide, karaya, or a combination thereof.

Any suitable amount of polymer 9 can be employed, provided the amount of polymer 9 effectively provides structure and strength to the pressure sensitive adhesive 14, provides structure and strength to the cosmetic formulation 5, and/or provides adhesiveness to the adhesive skin patch 1; and the effective amount of polymer 9 remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Typically, the suitable amount of polymer 9 will depend upon the specific polymer 9 employed. Specifically, karaya can be employed as the polymer 9 up to about 60 wt. % of the cosmetic formulation 5, in about 5.0 wt. % to about 45 wt. % of the cosmetic formulation 5, or in about 8.0 wt. % to about 40 wt. % of the cosmetic formulation 5; polyacrylamide can be employed as the polymer 9 up to about 40 wt. % of the cosmetic formulation 5, in about 5.0 wt. % to about 35 wt. % of the cosmetic formulation 5, or in about 8.0 wt. % to about 30 wt. % of the cosmetic formulation 5; or both karaya and polyacrylamide can be employed as the polymer 9, wherein karaya is present in about 5.0 wt. % to about 35 wt. % of the cosmetic formulation 5 and polyacrylamide is present in about 5.0 wt. % to about 30 wt. % of the cosmetic formulation 5.

Emulsifier

The cosmetic formulation 5 or pressure sensitive adhesive 14 can optionally include an emulsifier 16 (i.e., compound that emulsifies the cosmetic formulation 5 or the pressure sensitive adhesive 14). One suitable emulsifier 16 is pectin. The emulsifier 16 (e.g., pectin) can be present in any suitable amount, provided the suitable amount is effective to emulsify the cosmetic formulation 5 or the pressure sensitive adhesive 14 and the emulsifier 16 remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Specifically, the emulsifier 16 (e.g., pectin) can be present up to about 30.0 wt. % of the cosmetic formulation 5, in about 1.0 wt. % to about 20.0 wt. % of the cosmetic formulation 5, or in about 2.0 wt. % to about 10.0 wt. % of the cosmetic formulation 5.

Humectant

The cosmetic formulation 5 can optionally include one or more humectants 17 to provide a moistening effect to the pressure sensitive adhesive 14. The humectant 17 can optionally hydrate the polymer 9. Any suitable humectant 17 can be employed, provided the humectant 17 effectively provides a moistening effect to the pressure sensitive adhesive 14 and the humectant 17 remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. One suitable humectant 17 is glycerin. Other suitable humectants 17 include polyhydric alcohols such as ethylene glycol, propylene glycol, triethylene glycol, tetraethylene glycol, sorbitol, and combinations thereof.

Any suitable amount of humectant 17 can be employed, provided the amount of humectant 17 effectively provides a moistening effect to the pressure sensitive adhesive 14 and the amount of humectant 17 effectively remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Typically, the suitable amount of humectant 17 will depend upon the specific humectant 17 employed and the specific polymer 9 employed. For example, karaya, polyacrylamide, or a combination thereof can be employed as the polymer 9 and glycerin can be employed as the humectant 17, wherein the glycerin is present in about 25.0 wt. % to about 70.0 wt. % or in about 40.0 wt. % to about 55.0 wt. % of the cosmetic formulation 5.

Filler

The cosmetic formulation 5 can optionally include one or more fillers 6. Any suitable filler 6 can be employed. Suitable fillers 6 include malto dextrin, dextrin, 70% sorbitol water, modified starches, depolymerized starches, and methylcellulose. As used herein, “malto dextrin” is a dextrose equivalent, wherein dextrose is D-glucose. Malto dextrin is commercially available as Amaizo Lodex 5 from American Maize-Products (Hammond, Ind.). Any suitable amount of filler can be employed in the cosmetic formulation 5. The suitable amount of filler can depend in part upon the specific filler present in the cosmetic formulation 5. For example, malto dextrin can be present up to about 20.0 wt. % of the cosmetic formulation 5, or in about 1.0 wt. % to about 10.0 wt. % of the cosmetic formulation 5.

Fragrance

The cosmetic formulation 5 can optionally include a fragrance 19. As used herein, a “fragrance” refers to substance having an odor or a scent detectable to a mammal (e.g., human). Specifically, the fragrance 19 can be a floral scent, a food scent, a fruit scent, a plant leaf scent, or any combination thereof. The fragrance 19 will preferably have a low to moderate volatility, so that its evaporation from the adhesive patch 1 is rendered minimal to moderate. The volatility will, however, be high enough such that when desirable, the odor or scent can be detected by the patient. Preferably, when the fragrance 19 is employed, the cosmetic formulation 5 of the adhesive patch 1 will emit an odor or scent that is detected by the patient for a period of at least about 12 hours, at least about 8 hours, at least 4 hours, or at least about 2 hours.

The fragrance 19 can be a low odor fragrance, a high odor fragrance, or a mixture thereof. As used herein, a high odor fragrance 19 can effectively mask the odor of another component, such as the cosmetic agent 15. As used herein, a low odor fragrance 19 can partially mask the odor of another component, such as the cosmetic agent 15. Some people, especially adults, have shown a preference for cosmetic formulations 5 wherein the odor of the cosmetic agent 15 is not detectable. These cosmetic formulations 5 can include a fragrance 19 that possesses an odor higher than the cosmetic agent 15. In such situations, the odor of the cosmetic agent 15 will be less noticeable or detectable than the odor of the fragrance 19 because the fragrance 19 will substantially completely mask the odor of the cosmetic agent 15.

It is appreciated that the suitable fragrances 19 would be known to those skilled in the art. It is also appreciated that those skilled in the art understand that suitable fragrances 19 are commercially available from, for example, Alpine Aromatics (Piscataway, N.J.), Andrea Aromatics (Princeton, N.J.), Arylessence, Inc. (Marietta, Ga.), Belmay Co., Inc. (Yonkers, N.Y.), Crami Flavor & Fragrance Co., Inc. (City of Commerce, Calif.), Creative Fragrances Mfgr. Inc. (Dallas, Tex.), Drom International Co. (Tawaco, N.J.), Fleurchem, Inc. (Middletown, N.Y.), Great Lakes Chem. Corp. (Lafayette, Ind.), Kraus & Co., Inc. (Battle Creek, Mich.), The Lebermuth Co., Inc. (Mishawaka, Ind.), Penta Manufacturing (Livingston, N.J.), Shaw Mudge & Co. (Shelton, Conn.), Synarome Corp. (NY, N.Y.), Penreco (Houston, Tex.), Tracy Chemical Co. (Portland, Oreg.), Belle-Aire Fragrances (Mundelein, Ill.), Gusta Fragrances Co. (Chesire, Conn.), Atlanta Fragrance (Kennesaw, Ga.), and Bell Flavors & Fragrances, Inc (Northbrook, Ill.). It is further appreciated that those of skill in the art understand that the fragrances can be any suitable compound or compounds commercially available from, for example, the above vendors, or any combination thereof.

As the number of suitable fragrances 19 is too voluminous and expansive to exhaustively list herein, suitable exemplary fragrances 19 are disclosed herein. Suitable exemplary fragrances 19 include, e.g., grape fragrance, musk fragrance, light vanilla fragrance, Jergens lotion fragrance, Vaseline Intensive Care fragrance, Nivea Lotion fragrance, Ivory Soap fragrance, amaretto fragrance, blueberry fragrance, coffee fragrance, egg nog fragrance, peanut butter fragrance, rum cake fragrance, honey almond fragrance, ginger bread house fragrance, coffee cake & spice fragrance, raspberry rose fragrance, cucumber, sassafras fragrance, strawberry fragrance, grapefruit pink fragrance, home sweet fragrance, jeweled citrus fragrance, lemon, mango fragrance, mulberry fragrance, orange flower fragrance, passion fruit fragrance, pikaki fragrance, freesia fragrance, china rain fragrance, coconut fragrance, apple fragrance, baked bread fragrance, cornucopia fragrance, lemon chiffon fragrance, peppermint twist fragrance, white cake fragrance, cherry pie fragrance, sugar plum fragrance, plum fragrance, romantic fragrance, sea fresh fragrance, tea fragrance, green floral fragrance, honeydew fragrance, kiwi fragrance, lilac fragrance, may bouquet fragrance, neutralizer fragrance, patchouli fragrance, peach fragrance, pine apple blossom fragrance, chocolate mint fragrance, frankincense fragrance, baked apple pie fragrance, cappuccino fragrance, cran-apple fragrance, maple syrup fragrance, buttered popcorn fragrance, sugar cookie fragrance, cotton candy fragrance, cranberry cobbler fragrance, plumeria fragrance, rum fragrance, spring fever fragrance, watermelon fragrance, guava fragrance, honeysuckle fragrance, hyacinth fragrance, macadamia nut fragrance, melon fragrance, oakmoss fragrance, papaya fragrance, pear pineapple fragrance, blueberry fragrance, citrus-ginseng fragrance, garden dreams fragrance, banana creme pie fragrance, chocolate mint fragrance, cranberry fragrance, macadamia nut fragrance, pumpkin pie fragrance, chocolate German cake fragrance, banana nut bread fragrance, sweet potato pie fragrance, raspberry fragrance, sandalwood fragrance, spring flowers fragrance, ylang fragrance, heather fragrance, jasmine fragrance, lavender fragrance, magnolia fragrance, mountain air fragrance, orange essence fragrance, paradise fragrance, peony fragrance, alpine breeze fragrance, chamomile fragrance, clover fragrance, gardenia fragrance, or any combination thereof.

The fragrance 19 can be identifiable or unidentifiable. An unidentifiable fragrance 19 has an odor that cannot be readily identified by the average consumer. An unidentifiable fragrance 19 does have an odor, but the odor cannot be readily identified by the average consumer. A minimally odorous fragrance 19 is one without any distinct odor. It is not odor-free but the odor is not recognizable and exceedingly faint.

The fragrance 19 can be produced from a single compound or from a mixture of two or more compounds. As such, suitable fragrances 19 can include (a) at least one low odor fragrance 19, (b) at least one high odor fragrance 19, or (c) a combination of at least one low odor fragrance 19 and at least one high odor fragrance 19. Any suitable combination of low odor fragrance 19, high odor fragrance 19, or combination thereof can be employed.

The fragrance 19 can be present in any suitable and effective amount, e.g., up to about 10 wt/% of the cosmetic formulation 5, up to about 1.0 wt. % of the cosmetic formulation 5, or up to about 0.1 wt. % of the cosmetic formulation 5.

Skin Protectant or Skin Conditioner

The cosmetic formulation 5 can optionally include a skin protectant 18 (i.e., topical moisturizer or skin conditioner). Any suitable skin protectant 18 can be employed, provided the skin is effectively protected or moisturized and the skin protectant remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Additionally, it is preferable that the skin conditioner is pharmaceutically acceptable for topical use in humans.

Suitable topical moisturizers 18 include, e.g. an alpha hydroxy acid, a glycosaminoglycan, grape seed oil, cranberry seed oil, green tea, white tea, methylparaben, propylparaben, caffeine, xanthine, Vitamin B-3, nicotinamide, licorice, calamine, aluminum hydroxide gel, cocoa butter, aloe, lanolin, glycerin, Vitamin E, Vitamin E acetate, farnesol, glycyrrhetinic acid, propylene glycol, ethylene glycol, triethylene glycol, hard fat, kaolin, lanolin, mineral oil, petrolatum, topical starch, white petroleum, cod liver oil, shark liver oil, zinc oxide; or a combination thereof. Additional suitable topical moisturizers 18 are disclosed, e.g., in U.S. Pat. Nos. 6,096,334; 6,096,033; 5,741,510; 5,536,263; 4,675,009; 4,307,717; 4,274,420; 5,976,565; 5,536,263; and references cited therein.

As used herein, “aluminum hydroxide gel” refers to a suspension containing aluminum oxide (Al₂O₃), mainly in the form of a hydroxide. It is typically obtained by drying the product of interaction in aqueous solution of an aluminum salt with ammonium or sodium carbonate.

As used herein, “cocoa butter” refers to a fatty substance in cocoa beans; a thick oily solid obtained from cocoa beans and used in making chocolate, cosmetics, and suntan oil. Also known as threobroma oil, it lubricates and softens the skin.

As used herein, “topical starch” refers to cornstarch.

As used herein, “kaolin” refers to aluminum silicate; powdered and freed from gritty particles by elutriation. Kaolin refers to the name of the locality in China where the substance is found in abundance.

As used herein, “white petroleum” refers to a purified mixture of hydrocarbons obtained from petroleum. A bleached version of yellow soft paraffin, it is used as an emollient and as a base for ointments. It is odorless when rubbed into the skin and not readily absorbed.

As used herein, “mineral oil” refers to the heavy liquid petrolatum; liquid paraffin or petroleum; a mixture of liquid hydrocarbons obtained from petroleum, and is typically used as a vehicle in pharmaceutical preparations.

As used herein, “petrolatum” refers to petroleum jelly; a yellow soft paraffin; a yellowish mixture of the softer members of the paraffin or methane series of hydrocarbons, obtained from petroleum as an intermediate product in the distillation; typically used as a soothing application to bums and abrasions of the skin, and as a base for ointments.

As used herein, “cod liver oil” refers to the partially destearinated fixed oil extracted from the fresh livers of Gadus morrhuae and other species of the family Gadidae, containing Vitamins A and D.

As used herein, “shark liver oil” refers to the oil extracted from the livers of sharks, mainly of the species Hypoprion brevirostris; a rich source of Vitamins A and D.

As used herein, “zinc oxide” refers to ZnO, which is typically used as a protective ointment.

As used herein, “calamine” is a pink powder of zinc oxide and a skin protectant containing about 98% zinc oxide and about 0.5% ferric oxide; “aloe” is the dried latex of leaves of Curaco Aloe (Aloe barbadenis Miller, Aloe vera Linne) or Cape Aloe (Aloe ferox Miller and hybrids), of the family Liliacaea. Aloe is commercially available as Aloe Vera Gel from Terry Laboratories (Melbourne, Fla.). Aloe Vera Gel is commercially available as Aloe Vera Gel 40× (20.0 wt. % solution in water), Aloe Vera Gel 1× (0.5 wt. % solution in water), Aloe Vera Gel 10× (5.0 wt. % solution in water), or solid Aloe Vera. The solid Aloe Vera can be dissolved in a carrier, such as water, to the desired concentration. In addition, the commercially available forms of Aloe Vera are optionally available as decolorized Aloe Vera.

As used herein, “Vitamin E” is 3,4-dihydro-2,5,7,8-tetramethyl-2-(4,8,12-trimethyltridecyl)-2H-1-benzopyran-6-ol; “Vitamin E acetate” is 3,4-dihydro-2,5,7,8-tetramethyl-2-(4,8,12-trimethyltridecyl)-2H-1-benzopyran-6-ol acetate; “lanolin” is the fat-like secretion of the sebaceous glands of sheep (i.e., complex mixture of esters and polyesters of 33 high molecular weight alcohols and 36 fatty acids) which is deposited onto the wool fibers; “farnesol” is 3,7,11-trimethyl-2,6,10-dodecatrien-1-ol. Farnesol is commercially available from American Radiolabeled Chemicals (ARC) (St. Louis, Mo.), and “glycyrrhetinic acid” is a pentacyclic triterpenoid derivative of the beta-amyrin type and is shown below:

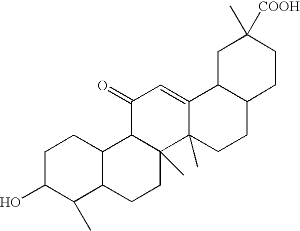

Any suitable amount of skin protectant 18 can be employed, provided the suitable amount of skin protectant 18 effectively protects or moisturizes the skin and the effective amount of skin protectant 18 remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Additionally, it is preferable that the amount of skin conditioner employed is pharmaceutically acceptable for topical use in humans.

Specifically, the skin protectant 18 can be present up to about 20.0 wt. %, up to 10.0 wt. %, up to 5.0 wt. %, or up to 2.0 wt. % of the cosmetic formulation 5. The suitable and effective amount of skin protectant 18 will depend in part upon the specific skin protectant 18 present in the cosmetic formulation 5. For example, Aloe Vera Gel, 10× can be present up to about 20.0 wt. % of the cosmetic formulation 5, up to about 10.0 wt. % of the cosmetic formulation 5, up to about 5.0 wt. % of the cosmetic formulation 5, or up to about 1.0 wt. % of the cosmetic formulation 5. In addition, Vitamin E acetate can be present up to about 10.0 wt. % of the cosmetic formulation 5, up to about 5.0 wt. % of the cosmetic formulation 5, up to about 3.0 wt. % of the cosmetic formulation 5, up to about 2.0 wt. % of the cosmetic formulation 5, or up to about 1.0 wt. % of the cosmetic formulation 5. Preferably, the skin conditioner will be present in an amount that is consistent with any State or Federal regulations.

The skin protectant 18 can be located in at least a portion of the front side 3 of the backing 2, on at least a portion of the front side 3 of the backing 2, or on and in at least a portion of the front side 3 of the backing 2. As such, the skin protectant 18 can be located on the entire surface of the front side 3 of the backing 2 or the skin protectant 18 can be located on a portion of the surface of the front side 3 of the backing 2. Preferably, the skin protectant 18 can be located on the entire surface of the front side 3 of the backing 2. In addition to being located on the surface of the front side 3 of the backing 2, the skin protectant 18 can be located in at least a portion of the underlying surface of the front side 3 of the backing 2 (i.e., the skin protectant 18 can be partially embedded into the backing 2). As shown in FIG. 10, the skin protectant 18 can penetrate a substantial portion of the front side 3 of the backing 2, as disclosed, e.g., in U.S. Pat. No. 5,536,263, and references cited therein. For example, the skin protectant 18 can penetrate about one-tenth to about nine-tenths the thickness of the backing 2, or about one-fourth to about nine-tenths the thickness of the backing 2. As such, the skin protectant 18 can be partially embedded into the backing 2. Preferably, the skin protectant 18 can be located on the entire front side 3 of the backing 2 and partially in the front side 3 of the backing 2 (i.e., the skin protectant 18 is partially embedded into the backing 2). Alternatively, a portion of the front side 3 of the backing 2 can include the skin protectant 18 and other portions of the front side 3 of the backing 2 can include any combination of the solvent 13, pressure sensitive adhesive 14, and cosmetic agent 15. When the adhesive skin patch 1 is placed upon the skin of a patient (e.g., human), the skin protectant 18 can be in continuous contact with the skin surface of the patient.

Preservative

The cosmetic formulation 5 can optionally include a preservative 7 that is useful for preventing bacterial growth, mold growth, fermentation, and/or decomposition. As used herein, “preservative” refers to any substance which prevents bacterial growth, mold growth, fermentation, and/or decomposition. Concise Chemical and Technical Dictionary, 4th enlarged edition, Chemical Publishing Co., Inc., NY, N.Y. p. 939 (1986). Any suitable preservative 7 can be employed, provided the preservative 7 effectively prevents bacterial growth, mold growth, fermentation, and/or decomposition; and the preservative 7 remains stable in the cosmetic formulation 5. Preferably, the stability is over a prolonged period of time, e.g., up to about 2 years, up to about 1 year, or up to about 6 months, typically experienced in the manufacturing, packaging, shipping, and/or storage of the adhesive skin patch 1.

Suitable preservatives 7 include, e.g., sodium meta bisulfite (NaS₂O₅), sodium bisulfite (NaHSO₃), quat-15, parabens, dichlorobenzyl alcohol, ethylene diamine tetreacetic acid, formaldehyde, gum benzoin, imidazolidinyl urea, phenylmercuric acetate, poly aminopropyl biguanide, proply gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazol-compound, chloromethyl-methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chlorohexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosol, and pharmaceutically acceptable salts thereof. Preferably, the preservative is quat-15, which is commercially available from Dow Chemical (Midland Mich.); methyl paraben; ascorbic acid; or a combination thereof.

The preservative 7 can be employed in any suitable amount provided the amount of preservative 7 effectively prevents bacterial growth, mold growth, fermentation, and/or decomposition and the effective amount of preservative 7 remains stable in the cosmetic formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. The preservative 7 can be present up to about 99.9 wt. % of the cosmetic formulation 5, up to about 20.0 wt. % of the cosmetic formulation 5, up to 5.0 wt. % of the cosmetic formulation 5, or up to 1.5 wt. % of the cosmetic formulation 5. The amount of preservative 7 present in the cosmetic formulation 5 will typically depend upon the specific compound or compounds employed as the preservative 7. For example, quat-15 can be employed in about 0.01 wt. % to about 1.5 wt. % of the cosmetic formulation 5, in about 0.05 wt. % to about 0.15 wt. % of the cosmetic formulation 5, or in about 0.08 wt. % to about 0.12 wt. % of the cosmetic formulation 5.

Astringent

The cosmetic formulation 5 can optionally include an astringent 20. As used herein, an “astringent” refers to a substance that causes tissue (e.g., skin) to contract. Mosby's Medical Encyclopedia, CD-Rom version 2.0 (1997); and Mosby's Medical, Nursing, & Allied Health Dictionary, Kenneth Anderson, 5th Ed., St. Louis, Mo. (1998). The astringent 20 can be employed to contract the skin surface of the mammal (e.g., human) thereby firming the skin surface. Any suitable astringent 20 can be employed, provided the astringent 20 effectively causes skin to contract. Preferably, the astringent 20 is pharmaceutically acceptable for topical use in humans. Suitable astringents 20 include, e.g., alum, tannic acid, calamine, witch hazel, zinc oxide, or a combination thereof.

As used herein, “with hazel” refers to hamamelis; “alum” refers to a double sulfate salt of aluminum and a monovalent metal that is typically used as an astringent in lotions and douches; and “tannic acid” refers to a group of compounds (tannins) with astringent taste obtained from the bark, fruits, and leaves of many plants (e.g., bark of oak).

Any suitable and effective amount of astringent 20 can be employed, provided the astringent 20 effectively causes skin tissue to contract. The amount of astringent 20 employed can be up to about 40 wt. % of the of the cosmetic formulation 5, or up to about 25 wt. % of the thetapeutic formulation 5. The amount of astringent 20 employed will typically depend upon the specific astringent or astringents employed. Specifically, calamine can be present up to about 25 wt. % of the cosmetic formulation 5; witch hazel can be present up to about 50 wt. % of the cosmetic formulation 5; and/or zinc oxide can be present up to about 25 wt. % of the cosmetic formulation 5. More specifically, calamine can be present in about 5 wt. % to about 25 wt. % of the cosmetic formulation 5; witch hazel can be present in about 10 wt. % to about 50 wt. % of the cosmetic formulation 5; and/or zinc oxide can be present in about 5 wt. % to about 25 wt. % of the cosmetic formulation 5.

Skin Absorption Enhancer

The cosmetic formulation 5 includes a skin absorption enhancer 21. As used herein, a “skin absorption enhancer” refers to any substance the aids, assists, and increases the ability of a substance (e.g., cosmetic agent 15) to be absorbed into the skin surface of a mammal (e.g., human).

Suitable skin absorption enhancers 21 include, e.g., diethylene glycol monoethyl ether (transcutol), dimethyl sulfoxide (DMSO), C₁₀DMSO, ionic surfactants, non-ionic surfactants, isopropyl myristate (IPM), and combinations thereof.

Specifically, the skin absorption enhancer 21 can include diethylene glycol monoethyl ether (transcutol). As used herein, “diethylene glycol monoethyl ether” or “transcutol” refers to 2-(2-ethoxyethoxy)ethanol [CAS NO. 001893]..

The skin absorption enhancer 21 can be present in any suitable, safe, and effective amount. Typically, the skin absorption enhancer 21 will be present up to about 99 wt. % of the cosmetic formulation 5, up to about 10 wt. % of the cosmetic formulation 5, up to about 1.0 wt. % of the cosmetic formulation 5, or up to about 0.1 wt. % of the cosmetic formulation 5.

Keratolytic Agent

The cosmetic formulation 5 can optionally include a keratolytic agent 22. As used herein, a “keratolytic agent” refers to a substance that causes desquamation (loosening) and debridement or sloughing of the surface cells of the epidermis. Any suitable keratolytic agent 22 can be employed, provided the keratolytic agent 22 effectively causes desquamation (loosening) and debridement or sloughing of the surface cells of the epidermis. Preferably, the keratolytic agent 22 is pharmaceutically acceptable for topical use in humans.

Suitable keratolytic agents 22 include, e.g., alcloxa, resorcinol, or a combination thereof. As used herein, “alcloxa” refers to Al-chlorhydroxy allontoinate; and “resorcinol” refers to m-dihydroxybenzene or 1,3-benzenediol.

Any suitable and effective amount of keratolytic agent 22 can be employed, provided amount of keratolytic agent 22 effectively causes desquamation (loosening) and debridement or sloughing of the surface cells of the epidermis. Preferably, the amount of keratolytic agent 22 is pharmaceutically acceptable for topical use in humans. The amount of keratolytic agent 22 will typically depend upon the specific keratolytic agent 22 or keratolytic agents 22 employed. Specifically, alcloxa can be present up to about 2.0 wt. % of the cosmetic formulation 5; and/or resorcinol can be present up to about 3.0 wt. % of the thetapeutic formulation 5. More specifically, alcloxa can be present in about 0.2 wt. % to about 2.0 wt. % of the cosmetic formulation 5; and/or resorcinol can be present in about 1.0 wt. % to about 3.0 wt. % of the thetapeutic formulation 5.

The cosmetic formulation 5 can preferably remain stable over the period of time typically experienced with the manufacturing, packaging, shipping, and/or storage of the adhesive skin patch 1, e.g., up to about a month, up to about a year, up to about two years, or up to about 3 years. The stability of the cosmetic agent 15, for example, is due in part to the cosmetic formulation 5 including the cosmetic agent 15 in an adhesive formulation. The adhesive formulation is preferably a hydrogel that holds the cosmetic agent 15 in an available form while maintaining the necessary stability, pressure sensitive adhesion and effectiveness over a prolonged period of time, e.g., up to about a month, up to about a year, up to about two years, or up to about 3 years.

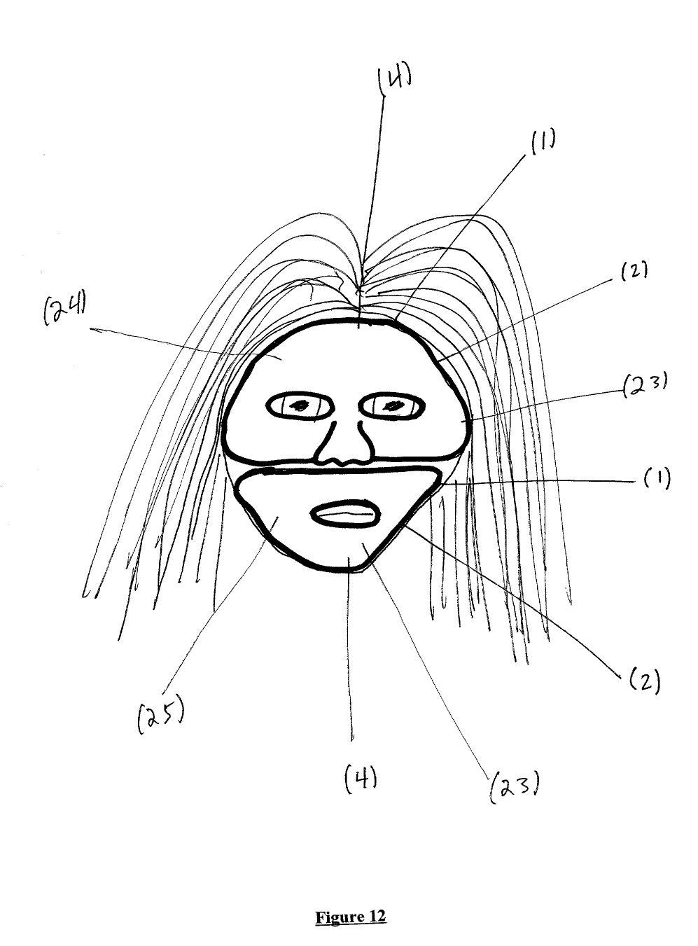

The adhesive skin patch 1 can have any suitable size and shape. In addition, the adhesive skin patch 1 can be cut, as desired, to provide an adhesive skin patch 1 of a desired size and shape. The adhesive skin patch 1 can be cut with any suitable cutting device such as a scissors, scalpel, or knife. In one specific embodiment of the present invention, the adhesive skin patch 1 can be a face mask, as shown in FIG. 11.

In one embodiment of the present invention, the adhesive skin patch 1 can be rectangular and can have a release liner 10 mounted on the front side 3 of the backing 2 of the adhesive skin patch 1. In such an embodiment, the adhesive skin patch 1 can typically have a length of up to about 10 inches, up to about 8 inches, up to about 5 inches, or up to about 3 inches. The adhesive skin patch 1 can have any suitable width. Typically, the adhesive skin patch 1 will have a width of up to about 10 inches, up to about 8 inches, up to about 5 inches, or up to about 3 inches. Additionally, the adhesive skin patch 1 can have any suitable thickness. Typically, the adhesive skin patch 1 will have a thickness of about 0.10 mm to about 2.0 mm, about 0.15 mm to about 1.0 mm, or about 0.20 mm to about 0.75 mm.

In one specific embodiment of the present invention, the adhesive skin patch 1 can be crescent, oval or circular in shape. The circular adhesive skin patch 1 can have a diameter of about 0.1 inch to about 10 inches. Preferably, the circular adhesive skin patch 1 can have a diameter of about 1.5 inches to about 5 inches. See, FIG. 7.

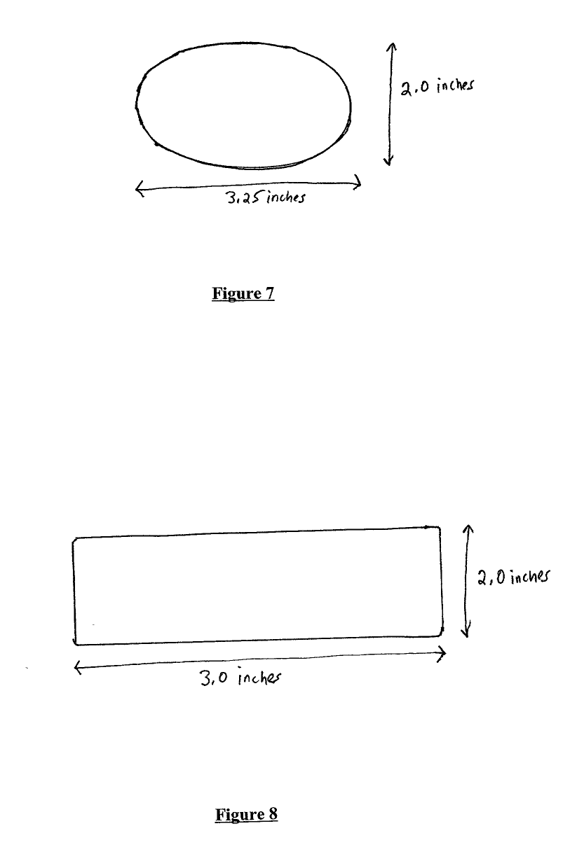

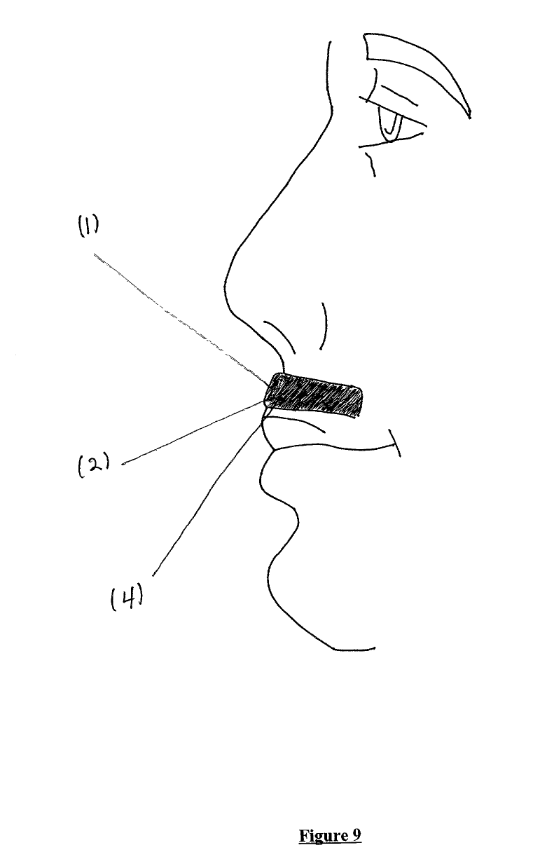

In another specific embodiment of the present invention, the adhesive skin patch 1 can be rectangular in shape. The rectangular adhesive skin patch 1 can have a length of about 1 inch to about 10 inches and a width of about 1 inch to about 10 inches. Preferably, the rectangular adhesive skin patch 1 can have a length of about 2 inches to about 5 inches and a width of about 2 inches to about 5 inches. See, FIG. 8.

In one embodiment of the present invention, the adhesive skin patch 1 can have a release liner 10 mounted on the front side 3 of the backing 2 of the adhesive skin patch 1. In such an embodiment, one or more adhesive skin patches 1 can be mounted on the release liner 10. For example, one adhesive skin patch 1 can have one release liner 10 mounted on the front side 3 of the backing 2 of the adhesive skin patch 1. Alternatively, about 2 to about 100 or about 2 to about 20 adhesive skin patches 1 can be mounted on the release liner 10. The cost of having two or more patches 1 on a single release liner 10 is typically less expensive than skin patches 1 that are separately mounted on a single release liner 10. In addition, some consumers may prefer the ease and comfort of carrying a single patch assembly that includes a single release liner 10 and more than one (e.g., about 2 to about 20, or about 2 to about 10) adhesive patches 1 mounted on the single release liner 10.

The adhesive skin patch 1 can be applied to the skin surface of a mammal (e.g., human). Suitable skin surfaces include, e.g., the face, neck, head, shoulder, chest, back, abdomen, leg, foot, arm, and hand regions. Specifically, the skin surface can include the face region.

The adhesive patch 1 of the present invention can be formulated or manufactured employing the above components. The adhesive patch 1 of the present invention can be formulated or manufactured using any suitable technique. Preferably, the adhesive patch 1 can be formulated or manufactured as described herein or as described in U.S. Pat. No. 5,536,263; U.S. Pat. No. 5,741,510; and references cited therein; wherein the oil premix includes the cosmetic agent 15, propylene glycol, and solvent 13; the glycerin premix includes glycerin, Vitamin E, and aloe vera gel; and the adhesive premix includes the adhesive, polymer 9, and water; and wherein the backing can be treated with a sizing agent 8 prior to the infusion of the cosmetic formulation 5.

The adhesive patch 1 of the present invention can be applied to a suitable skin surface of a mammal (e.g., human) for a suitable period of time. After a suitable period of time has elapsed, the adhesive patch 1 of the present invention can then be peeled off the skin surface. After a reasonable period of time has elapsed, another adhesive patch 1 of the present invention can be applied to the same skin surface.

In FIGS. 1-10, the presence of the components of the adhesive patch 1 of the present invention (e.g., cosmetic formulation 5) is indicated with a single number and a specific location. It is appreciated that those of skill in the art understand that those components can be present in locations other than those expressly shown. The express locations are shown for illustration purposes and are not intended to be limiting. Additionally, it is appreciated that those of skill in the art understand that the cosmetic formulation 5 will typically include the cosmetic agent 15, solvent 13 that dissolves the cosmetic agent 15, skin absorption enhancer 21, at least one of a pressure sensitive adhesive 14 and a polymer 9, and optionally one or more of a filler 6, preservative 7, sizing agent 8, emulsifier 16, humectant 17, skin protectant 18, fragrance 19, astringent 20, and keratolytic agent 22.

The adhesive patch 1 of the present invention can be in the form of an adhesive face mask 23, as shown in FIGS. 11 and 12. The adhesive face mask 23 can include a first portion 24 and a second portion 25. Each of the first portion 24 and second portion 25 will include a flexible backing 2 having a front side 3 and a back side 4 and a cosmetic formulation 5 positioned on at least a portion of the front side 3 of the backing 2, in at least a portion of the front side 3 of the backing 2, or on and in at least a portion of the front side 3 of the backing 2. The first portion 24 includes two apertures for the eyes of a person's face, such that when in use (see, FIG. 12), the front side 3 of the backing 2 adhesively attaches to a skin surface of the person's face near the person's eyes. The second portion includes an aperture corresponding to the mouth of a person's face, such that when in use (see, FIG. 12), the front side 3 of the backing 2 adhesively attaches to a skin surface of the person's face near the person's mouth. In one specific embodiment, the first portion 24 is contoured to the nose, cheeks and eyes of a person's face and the second portion 25 is contoured to the mouth and chin of a person's face.

The invention can be illustrated with the following non-limiting examples:

### EXAMPLES

### Example 1

| component | wt. % |
|---|---|
| poylacrylamide | 13.00 |
| glycerin | 53.50 |
| water | 19.00 |
| Vitamin A palmitate | 0.25 |
| Grapeseed oil | 0.50 |
| Fragrance | 0.25 |
| Ammonium lactate | 1.00 |
| Propylene glycol | 4.00 |
| Diethylene glycol ethyl | 5.00 |
| ether | |
| Emulsion adhesive | 3.00 |
| preservative | 0.50 |

### Example 2

| component | wt. % |
|---|---|
| karaya | 15.00 |
| glycerin | 51.50 |
| water | 19.00 |
| lycopene | 0.25 |
| Grapeseed oil | 0.50 |
| Fragrance | 0.25 |
| Vitamin E | 1.00 |
| Propylene glycol | 4.00 |
| Diethylene glycol ethyl | 5.00 |
| ether | |
| Emulsion adhesive | 3.00 |
| preservative | 0.50 |

### Example 3

| component | wt. % |
|---|---|
| poylacrylamide | 13.00 |
| glycerin | 52.50 |
| water | 19.00 |
| Kinetin extract | 0.25 |
| Grapeseed oil | 0.50 |
| Fragrance | 0.25 |
| Vitamin C | 1.00 |
| Propylene glycol | 4.00 |
| Diethylene glycol ethyl | 5.00 |
| ether | |
| Emulsion adhesive | 3.00 |
| Tartaric acid | 1.00 |
| Preservative | 0.50 |

### Example 4

| component | wt. % |
|---|---|
| gelatin | 10.00 |
| Polyvinyl alcohol | 4.00 |
| Polyacrylic acid | 6.00 |
| glycerin | 20.00 |
| water | 45.00 |
| Vitamin A palmitate | 0.25 |
| Grapeseed oil | 0.50 |
| Fragrance | 0.25 |
| Alcloxa | 1.00 |
| Propylene glycol | 4.00 |
| Propylene carbonate | 5.00 |
| Ammonium lactate | 3.00 |
| Triethanol amine | 1.00 |

### Example 5

| component | wt. % |
|---|---|
| Guar | 10.00 |
| Polyvinyl alcohol | 4.00 |
| Polyacrylic acid | 6.00 |
| glycerin | 20.00 |
| water | 45.00 |
| Vitamin A palmitate | 0.25 |
| Grapeseed oil | 0.50 |
| Fragrance | 0.25 |
| Vitamin C | 1.00 |
| Propylene glycol | 4.00 |
| Diethylene glycol ethyl | 5.00 |
| ether | |
| Emulsion adhesive | 3.00 |
| Borax | 1.00 |

### Example 6

| component | wt. % |
|---|---|
| Calcium Alginate | 10.00 |
| Pectin | 4.00 |
| Polyacrylic acid | 6.00 |
| glycerin | 20.00 |
| water | 45.00 |
| Vitamin E | 0.25 |
| Grapeseed oil | 0.50 |
| Fragrance | 0.25 |
| Vitamin C | 1.00 |
| Propylene glycol | 4.00 |
| Diethylene glycol ethyl | 5.00 |
| ether | |
| Emulsion adhesive | 3.00 |
| Triethanol amine | 1.00 |

### Example 7

| component | wt. % |
|---|---|
| gelatin | 10.00 |
| Polyvinyl pyrrolidone | 4.00 |
| Polyacrylic acid | 6.00 |
| glycerin | 20.00 |
| water | 45.00 |
| Vitamin E | 0.25 |
| Grapeseed oil | 0.50 |
| Fragrance | 0.25 |
| Vitamin C | 1.00 |
| Propylene glycol | 4.00 |
| Diethylene glycol ethyl | 5.00 |
| ether | |
| Emulsion adhesive | 3.00 |
| Triethanol amine | 1.00 |

### Example 8

| component | wt. % |
|---|---|
| gelatin | 10.00 |
| Polyvinyl alcohol | 5.00 |
| Polyacrylic acid | 6.00 |
| glycerin | 20.00 |
| water | 43.50 |
| caffeine | 2.00 |
| Grapeseed oil | 0.50 |
| Fragrance | 0.25 |
| Vitamin C | 1.00 |
| Propylene glycol | 3.75 |
| Diethylene glycol ethyl | 5.00 |
| ether | |
| Tartaric acid | 2.00 |
| Triethanol amine | 1.00 |

### Example 9

| component | wt. % |
|---|---|
| Calcium alginate | 10.00 |
| Polyvinyl alcohol | 4.00 |
| Starch polyacrylic acid graft | 6.00 |
| glycerin | 20.00 |
| water | 45.00 |
| Vitamin E | 0.25 |
| Grapeseed oil | 0.50 |
| Fragrance | 0.25 |
| Vitamin C sodium phosphate | 1.00 |
| Propylene glycol | 4.00 |
| Diethylene glycol ethyl ether | 5.00 |
| Emulsion adhesive | 3.00 |
| Triethanol amine | 1.00 |

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. An adhesive patch comprising a flexible backing having a front side and a back side and a cosmetic formulation positioned on at least a portion of the front side of the backing, in at least a portion of the front side of the backing, or on and in at least a portion of the front side of the backing; wherein the cosmetic formulation comprises:
a cosmetic agent;
a solvent;
a skin absorption enhancer; and
at least one of a pressure sensitive adhesive and a polymer.

2. The adhesive patch of claim 1 wherein the backing comprises a nonwoven fabric.

3. The adhesive patch of claim 1 wherein the backing comprises polycellulose fibers, polyester fibers, polyurethane fibers, polyolefin fibers, polyamide fibers, cotton fibers, or any mixture thereof.

4. The adhesive patch of claim 1 wherein the backing comprises open cell foam.

5. The adhesive patch of claim 4 wherein the open cell foam comprises polyurethane, polyvinyl chloride, polyethylene, or a combination thereof.

6. The adhesive patch of claim 1 wherein upon contact with skin, the backing retains the cosmetic formulation and the patch allows moisture from the skin to pass.

7. The adhesive patch of claim 1 wherein the cosmetic agent is an anti-oxidant.

8. The adhesive patch of claim 7 wherein the anti-oxidant is a free radical scavenger.

9. The adhesive patch of claim 8 wherein the free radical scavenger is at least one of lycopene, tumeric, green tea, white tea, an alpha hydroxy acid, beta hydroxy acid, Vitamin C, Vitamin E, Vitamin A, an acceptable salt thereof, or an acceptable derivative thereof.

10. The adhesive patch of claim 9 wherein the acceptable salt is an ammonium salt.

11. The adhesive patch of claim 9 wherein the alpha hydroxy acid is lactic acid, tartaric acid, citric acid, glycolic acid, malic acid, alpha-hydroxy octanoic acid, alpha-hydroxy caprylic acid, a mixed fruit acid, a sugar cane extract, or an acceptable salt thereof.

12. The adhesive patch of claim 11 wherein the acceptable salt is an ammonium salt.

13. The adhesive patch of claim 9 wherein the beta hydroxy acid is salicylic acid, beta-hydroxybutanoic acid, tropic acid, trethocanic acid, or an acceptable salt thereof.

14. The adhesive patch of claim 13 wherein the acceptable salt is an ammonium salt.

15. The adhesive patch of claim 1 wherein the cosmetic agent is a collagen synthesis stimulator, a fibroblast growth stimulator, a collagen cross-linking inhibitor, or a combination thereof.

16. The adhesive patch of claim 15 wherein the collagen synthesis stimulator is a plant extract containing kinetin (6 furfurylaminopurine), vitamin C, a copper containing peptide, or a combination thereof.

17. The adhesive patch of claim 15 wherein the fibroblast growth stimulator is a copper containing peptide, retin A, or a cytokine.

18. The adhesive patch of claim 17 wherein the cytokine is a Fibroblast Growth Factor.

19. The adhesive patch of claim 15 wherein the collagen cross-linking inhibitor is aminoguanidine or carnosine.

20. The adhesive patch of claim 1 wherein the cosmetic agent is tourmaline, caffeine, theophyline, or a combination thereof.

21. The adhesive patch of claim 1 wherein the cosmetic agent is present in about 0.01 wt. % to about 4.0 wt. % of the cosmetic formulation.

22. The adhesive patch of claim 1 wherein the cosmetic agent is present in about 0.05 wt. % to about 2.5 wt. % of the cosmetic formulation.

23. The adhesive patch of claim 1 wherein the cosmetic agent is present in about 0.1 wt. % to about 1.0 wt. % of the cosmetic formulation.

24. The adhesive patch of claim 1 wherein the cosmetic agent is located on the entire surface of the front side of the backing.

25. The patch of claim 1 wherein the cosmetic agent is completely embedded in the backing.

26. The adhesive patch of claim 1 wherein the solvent comprises a polyhydric alcohol, water, or a combination thereof.

27. The adhesive patch of claim 26 wherein the polyhydric alcohol is glycerin, propylene glycol, ethylene glycol, triethylene glycol, or a combination thereof.

28. The adhesive patch of claim 26 wherein the polyhydric alcohol is present up to about 90 wt. % of the cosmetic formulation.

29. The adhesive patch of claim 26 wherein the polyhydric alcohol is present in about 20 wt. % to about 60 wt. % of the cosmetic formulation.

30. The adhesive patch of claim 26 wherein the water is present in about 20 wt. % to about 80 wt. % of the cosmetic formulation.

31. The adhesive patch of claim 1 wherein the solvent is present in about 5 wt. % to about 90 wt. % of the cosmetic formulation.

32. The adhesive patch of claim 1 wherein the solvent comprises water; triethylene glycol; glycerin; propylene glycol; ethylene glycol; triacetin; 1,3-propane diol; 2-methyl-1,3-propane diol; glycerol ricinoleate; PEG-6 caprylic/capric glycerides; caprylic/capric triglycerides; propyleneglycol dicaprylate/dicaprate; glycerol monostearate; glycerol monocaprylate; glycerol monolaurate; neopentyl alcohol; 1-hexadecanol; hydroxypropyl beta-cyclodextrin; vitamin E; vitamin E acetate; deoxycholic acid; taurodeoxycholic acid; 3-[(3-cholamidopropyl) dimethylammonio]-1-propane-sulfonate; BigCHAP; cholic acid; cholesterol NF; propylene carbonate; lecithin; diethylene glycol ethyl ether; diethylene glycol ethyl ether acetate; an acceptable salt thereof; or a combination thereof.

33. The adhesive patch of claim 1 wherein the solvent is located on the entire surface of the front side of the backing.

34. The patch of claim 1 wherein the solvent is completely embedded in the backing.

35. The adhesive patch of claim 1 wherein the pressure sensitive adhesive comprises one or more acrylic ester copolymers.

36. The adhesive patch of claim 35 wherein each of the one or more acrylic ester copolymers is present up to about 30 wt. % of the cosmetic formulation.

37. The adhesive patch of claim 35 wherein each of the one or more acrylic ester copolymers is present in about 2 wt. % of the cosmetic formulation to about 30 wt. % of the cosmetic formulation.

38. The adhesive patch of claim 35 wherein all of the one or more acrylic ester copolymers, combined, are present in about 2 wt. % to about 30 wt. % of the cosmetic formulation.

39. The adhesive patch of claim 1 wherein the pressure sensitive adhesive is located on the entire surface of the front side of the backing.

40. The patch of claim 1 wherein the pressure sensitive adhesive is completely embedded in the backing.

41. The adhesive patch of claim 1 wherein the pressure sensitive adhesive further comprises an emulsifier.

42. The adhesive patch of claim 41 wherein the emulsifier is pectin.

43. The adhesive patch of claim 42 wherein the pectin is present in about 2.0 wt. % to about 20.0 wt. % of the cosmetic formulation.

44. The adhesive patch of claim 1 wherein the polymer is a polymer that includes a quaternary ammonium salt, gum tragacanth, gum Ghatti, gum agar, pectin, chitin, a derivative of chitin, carrageenan, a calcium cross-linked alginate, a cross-linked polymer by means of boron or a di- or tri-valent metal ion, an aldehyde cross-linked gelatin, gelatin, karaya, a polyacrylamide, a polyacrylic acid, xanthan gum, guar gum, a natural polymer, a synthetic polymer, a hydrophilic polymer, a hydrocolloidal polymer, starch, a starch derivative or graft copolymer, vinyl acetate copolymer, polyvinyl pyrrolidone, polyethylene oxide, algin, derivatives of algin, a polyacrylate, polymaleic acid, polymaleic anhydride, a polyurethane, a polyurea, gum acacia, locust bean gum, modified guar gum, maltodextrin, carboxymethyl cellulose, carboxypropyl cellulose, polyvinyl alcohol, poly AMPS, sodium polyacrylate, or a mixture thereof.

45. The adhesive patch of claim 1 wherein the polymer is polyvinyl alcohol, sodium polyacrylate, gelatin, or a combination thereof.

46. The adhesive patch of claim 45 wherein the polyvinyl alcohol is present in about 1.0 wt. % to about 20 wt. % the cosmetic formulation.

47. The adhesive patch of claim 45 wherein the sodium polyacrylate is present in about 1.0 wt. % to about 20.0 wt. % of the cosmetic formulation.

48. The adhesive patch of claim 45 wherein the gelatin is present in about 1.0 wt. % to about 30.0 wt. % of the cosmetic formulation.

49. The adhesive patch of claim 1 wherein the polymer is located on the entire surface of the front side of the backing.

50. The patch of claim 1 wherein the polymer is completely embedded in the backing.

51. The adhesive patch of claim 1 wherein the cosmetic formulation further comprises a fragrance.

52. The adhesive patch of claim 51 wherein the fragrance is a floral scent, a fruit scent, a plant leaf scent, a plant seed scent, a plant root scent, a plant berry scent, a plant bark scent, or any combination thereof.

53. The adhesive patch of claim 51 wherein the fragrance is grape fragrance, musk fragrance, light vanilla fragrance, Jergens lotion fragrance, Vaseline Intensive Care fragrance, Nivea Lotion fragrance, Ivory Soap fragrance, amaretto fragrance, blueberry fragrance, coffee fragrance, egg nog fragrance, peanut butter fragrance, rum cake fragrance, honey almond fragrance, ginger bread house fragrance, coffee cake & spice fragrance, raspberry rose fragrance, cucumber, sassafras fragrance, strawberry fragrance, grapefruit pink fragrance, home sweet fragrance, jeweled citrus fragrance, lemon, mango fragrance, mulberry fragrance, orange flower fragrance, passion fruit fragrance, pikaki fragrance, freesia fragrance, china rain fragrance, coconut fragrance, apple fragrance, baked bread fragrance, cornucopia fragrance, lemon chiffon fragrance, peppermint twist fragrance, white cake fragrance, cherry pie fragrance, sugar plum fragrance, plum fragrance, romantic fragrance, sea fresh fragrance, tea fragrance, green floral fragrance, honeydew fragrance, kiwi fragrance, lilac fragrance, may bouquet fragrance, neutralizer fragrance, patchouli fragrance, peach fragrance, pine apple blossom fragrance, chocolate mint fragrance, frankincense fragrance, baked apple pie fragrance, cappuccino fragrance, cran-apple fragrance, maple syrup fragrance, buttered popcorn fragrance, sugar cookie fragrance, cotton candy fragrance, cranberry cobbler fragrance, plumeria fragrance, rum fragrance, spring fever fragrance, watermelon fragrance, guava fragrance, honeysuckle fragrance, hyacinth fragrance, macadamia nut fragrance, melon fragrance, oakmoss fragrance, papaya fragrance, pear pineapple fragrance, blueberry fragrance, citrus-ginseng fragrance, garden dreams fragrance, banana creme pie fragrance, chocolate mint fragrance, cranberry fragrance, macadamia nut fragrance, pumpkin pie fragrance, chocolate German cake fragrance, banana nut bread fragrance, sweet potato pie fragrance, raspberry fragrance, sandalwood fragrance, spring flowers fragrance, ylang fragrance, heather fragrance, jasmine fragrance, lavender fragrance, magnolia fragrance, mountain air fragrance, orange essence fragrance, paradise fragrance, peony fragrance, alpine breeze fragrance, chamomile fragrance, clover fragrance, gardenia fragrance, or any combination thereof.

54. The adhesive patch of claim 1 wherein the cosmetic formulation is completely embedded in at least a portion of the front side of the backing.

55. The adhesive patch of claim 1 wherein the cosmetic formulation is located on the entire surface of the front side of the backing.

56. The adhesive patch of claim 1 wherein the backing is porous.

57. The adhesive patch of claim 1 wherein the backing is vapor permeable.

58. The adhesive patch of claim 1 wherein the backing comprises water insoluble material.

59. The adhesive patch of claim 1 wherein the backing comprises water soluble material.

60. The adhesive patch of claim 1 wherein the backing comprises polycellulose fibers, polyester fibers, polyurethane fibers, polyolefin fibers, polyamide fibers, cotton fibers, copolyester fibers, or any mixture thereof.

61. The adhesive patch of claim 1 wherein upon contact with skin, the backing retains the cosmetic formulation and the patch allows moisture from the skin to pass.

62. The adhesive patch of claim 1 wherein the backing has a thickness of about 0.025 mm to about 1.25 mm.

63. The adhesive patch of claim 1 wherein the backing comprises a nonwoven fabric.

64. The adhesive patch of claim 1 wherein at least a portion of the backing is treated with a sizing agent such that the portion of the backing that is treated with the sizing agent has a surface energy of about 20 dynes/cm² to about 65 dynes/cm².

65. The adhesive patch of claim 64 wherein the sizing agent is a fluorocarbon solution, a silicone-containing compound, or a combination thereof.

66. The adhesive patch of claim 65 wherein the backing that is treated with the fluorocarbon solution is Vilmed M1585 W/HY, Vilmed M1585H/HY, Vilmed M1586 W/HY, Vilmed M1586 H/HY, Vilmed M1570, Vilmed M1573 F, Vilmed M1573 FH, Vilmed M1577 F, Vilmed M1578 F, Vilmed M1578 FH, or a combination thereof.

67. The adhesive patch of claim 65 wherein the silicone-containing compound is a polydimethyl siloxane, a dialkylsiloxane, a dimethylsiloxo vinyl alkene, a dialkylsiloxo vinyl alkene, a dimethylsiloxo acrylate, a dialkylsiloxo acrylate, a vinyl terminated polydimethylsiloxane, a vinyl terminated polydialkylsiloxane, or a combination thereof.

68. The adhesive patch of claim 64 wherein the entire front side of the backing is treated with the sizing agent.

69. The adhesive patch of claim 64 wherein the sizing agent penetrates at least a portion of the underlying surface of the front side of the backing.

70. The adhesive patch of claim 64 wherein the sizing agent penetrates the entire underlying surface of the front side of the backing.

71. The adhesive patch of claim 64 wherein the entire backing is treated with the sizing agent.

72. The adhesive patch of claim 1 wherein the cosmetic formulation further comprises one or more skin conditioners.

73. The adhesive patch of claim 72 wherein the skin conditioner is an alpha hydroxy acid, a glycosaminoglycan, grape seed oil, cranberry seed oil, green tea, white tea, methylparaben, propylparaben, caffeine, xanthine, Vitamin B-3, nicotinamide, licorice, calamine, aluminum hydroxide gel, cocoa butter, aloe, lanolin, glycerin, Vitamin E, Vitamin E acetate, farnesol, glycyrrhetinic acid, propylene glycol, ethylene glycol, triethylene glycol, hard fat, kaolin, lanolin, mineral oil, petrolatum, topical starch, white petroleum, cod liver oil, shark liver oil, zinc oxide; or a combination thereof.

74. The adhesive patch of claim 73 wherein the alpha hydroxy acid is present up to about 5.0 wt. % of the cosmetic formulation.

75. The adhesive patch of claim 1 further comprising a keratolytic agent.

76. The adhesive patch of claim 75 wherein the keratolytic agent is alcloxa, resorcinol, or a combination thereof.

77. The adhesive patch of claim 76 wherein the alcloxa is present up to about 2.0 wt. % of the cosmetic formulation; or the resorcinol is present up to about 3.0 wt. % of the thetapeutic formulation.

78. The adhesive patch of claim 76 wherein the alcloxa is present in about 0.2 wt. % of the cosmetic formulation to about 2.0 wt. % of the cosmetic formulation; or the resorcinol is present in about 1.0 wt. % of the cosmetic formulation to about 3.0 wt. % of the thetapeutic formulation.

79. The adhesive patch of claim 1 wherein the cosmetic formulation further comprises a filler.

80. The adhesive patch of claim 76 wherein the filler is malto dextrin.

81. The adhesive patch of claim 80 wherein the malto dextrin is present in about 1.0 wt. % to about 10.0 wt. % of the cosmetic formulation.

82. The adhesive patch of claim 1 wherein the skin absorption enhancer is diethylene glycol monoethyl ether, dimethyl sulfoxide (DMSO), C₁₀DMSO, ionic surfactants, non-ionic surfactants, isopropyl myristate (IPM), or a combination thereof.

83. The adhesive patch of claim 1 wherein the skin absorption enhancer is diethylene glycol monoethyl ether.

84. The adhesive patch of claim 1 having a thickness of about 0.20 mm to about 0.75 mm.

85. The adhesive patch of claim 1 further comprising a release liner that is mounted on the front side of the backing.

86. The adhesive patch of claim 85 wherein more than one patch is mounted on the release liner.

87. The adhesive patch of claim 85 wherein about 2 to about 20 adhesive patches are mounted on the release liner.

88. The adhesive patch of claim 1 further comprising a preservative.

89. The adhesive patch of claim 88 wherein the preservative is sodium meta bisulfite (NaS₂O₅), sodium bisulfite (NaHSO₃), quat-15, parabens, dichlorobenzyl alcohol, ethylene diamine tetreacetic acid, formaldehyde, gum benzoin, imidazolidinyl urea, phenyl-mercuric acetate, poly aminopropyl biguanide, proply gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazol-compound, chloromethyl-methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chlorohexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosol, acceptable salts thereof; or a combination thereof.

90. The adhesive patch of claim 88 wherein the preservative is present in about 0.01 wt. % to about 1.5 wt. % of the cosmetic formulation.

91. The adhesive patch of claim 1 further comprising a keratolytic agent.

92. The adhesive patch of claim 91 wherein the keratolytic agent is alcloxa, resorcinol, or a combination thereof.

93. The adhesive patch of claim 91wherein the keratolytic agent is present in about 0.2 wt. % to about 3.0 wt. % of the cosmetic formulation.

94. The adhesive patch of claim 1 further comprising an astringent.

95. The adhesive patch of claim 94 wherein the astringent is alum, tannic acid, calamine, witch hazel, zinc oxide, or a combination thereof.

96. The adhesive patch of claim 94 wherein the astringent is present up to about 25 wt. % of the cosmetic formulation.

97. The adhesive patch of claim 1 further comprising a skin protectant or skin conditioner.

98. The adhesive patch of claim 97 wherein the skin protectant or skin conditioner is an alpha hydroxy acid, a glycosaminoglycan, grape seed oil, cranberry seed oil, green tea, white tea, methylparaben, propylparaben, caffeine, xanthine, Vitamin B-3, nicotinamide, licorice, calamine, aluminum hydroxide gel, cocoa butter, aloe, lanolin, glycerin, Vitamin E, Vitamin E acetate, farnesol, glycyrrhetinic acid, propylene glycol, ethylene glycol, triethylene glycol, hard fat, kaolin, lanolin, mineral oil, petrolatum, topical starch, white petroleum, cod liver oil, shark liver oil, zinc oxide; or a combination thereof.

99. The adhesive patch of claim 97 wherein the skin protectant or skin conditioner is present up to about 2.0 wt. % of the cosmetic formulation.

100. An adhesive patch comprising a flexible backing having a front side and a back side and a cosmetic formulation positioned on at least a portion of the front side of the backing, in at least a portion of the front side of the backing, or on and in at least a portion of the front side of the backing; wherein the cosmetic formulation comprises:
a cosmetic agent selected from the group consisting of: an antioxidant, a collagen synthesis stimulator, a fibroblast growth stimulator, a collagen cross-linking inhibitor, caffeine, theophyline, and combinations thereof;
a solvent;
a skin absorption enhancer; and
at least one of a pressure sensitive adhesive and a polymer.

101. An adhesive patch comprising a flexible backing having a front side and a back side and a cosmetic formulation positioned on at least a portion of the front side of the backing, in at least a portion of the front side of the backing, or on and in at least a portion of the front side of the backing; wherein the cosmetic formulation comprises:
a cosmetic agent selected from the group consisting of: Vitamin C, Vitamin E, Vitamin A, lactic acid, tartaric acid, citric acid, glycolic acid, malic acid, alpha-hydroxy octanoic acid, alpha-hydroxy caprylic acid, a mixed fruit acid, a sugar cane extract, salicylic acid, beta-hydroxybutanoic acid, tropic acid, trethocanic acid, a plant extract containing kinetin (6 furfurylaminopurine), Vitamin C, a copper containing peptide, retin A, a cytokine, a Fibroblast Growth Factor, aminoguanidine, carnosine, and combinations thereof;
a solvent;
a skin absorption enhancer; and
at least one of a pressure sensitive adhesive and a polymer.

102. An adhesive mask comprising:
a first portion and a second portion each comprising a flexible backing having a front side and a back side and a cosmetic formulation positioned on at least a portion of the front side of the backing, in at least a portion of the front side of the backing, or on and in at least a portion of the front side of the backing; wherein the cosmetic formulation comprises a cosmetic agent, a solvent, a skin absorption enhancer and at least one of a pressure sensitive adhesive and a polymer;
the first portion comprises two apertures for the eyes of a person's face, such that the front side of the backing adhesively attaches to a skin surface of the person's face near the person's eyes; and
the second portion comprises an aperture corresponding to the mouth of a person'face, such that the front side of the backing adhesively attaches to a skin surface of the person's face near the person's mouth.

103. An adhesive mask comprising:
a first portion and a second portion each comprising a flexible backing having a front side and a back side and a cosmetic formulation positioned on at least a portion of the front side of the backing, in at least a portion of the front side of the backing, or on and in at least a portion of the front side of the backing; wherein the cosmetic formulation comprises a cosmetic agent selected from the group consisting of: an antioxidant, a collagen synthesis stimulator, a fibroblast growth stimulator, a collagen cross-linking inhibitor, caffeine, theophyline, and combinations thereof;, a solvent, a skin absorption enhancer and at least one of a pressure sensitive adhesive and a polymer;
the first portion comprises two apertures for the eyes of a person's face, such that the front side of the backing adhesively attaches to a skin surface of the person's face near the person's eyes; and
the second portion comprises an aperture corresponding to the mouth of a person's face, such that the front side of the backing adhesively attaches to a skin surface of the person's face near the person's mouth.

104. An adhesive mask comprising:
a first portion and a second portion each comprising a flexible backing having a front side and a back side and a cosmetic formulation positioned on at least a portion of the front side of the backing, in at least a portion of the front side of the backing, or on and in at least a portion of the front side of the backing; wherein the cosmetic formulation comprises a cosmetic agent selected from the group consisting of: Vitamin C, Vitamin E, Vitamin A, lactic acid, tartaric acid, citric acid, glycolic acid, malic acid, alpha-hydroxy octanoic acid, alpha-hydroxy caprylic acid, a mixed fruit acid, a sugar cane extract, salicylic acid, beta-hydroxybutanoic acid, tropic acid, trethocanic acid, a plant extract containing kinetin (6 furfurylaminopurine), Vitamin C, a copper containing peptide, retin A, a cytokine, a Fibroblast Growth Factor, aminoguanidine, carnosine, and combinations thereof, a solvent, a skin absorption enhancer and at least one of a pressure sensitive adhesive and a polymer;
the first portion comprises two apertures for the eyes of a person's face, such that the front side of the backing adhesively attaches to a skin surface of the person's face near the person's eyes; and
the second portion comprises an aperture corresponding to the mouth of a person's face, such that the front side of the backing adhesively attaches to a skin surface of the person's face near the person's mouth.

105. An adhesive mask comprising:
a first portion and a second portion each comprising a flexible backing having a front side and a back side and a cosmetic formulation positioned on at least a portion of the front side of the backing, in at least a portion of the front side of the backing, or on and in at least a portion of the front side of the backing; wherein the cosmetic formulation comprises a cosmetic agent, a solvent, a skin absorption enhancer and at least one of a pressure sensitive adhesive and a polymer;
the first portion being contoured to the nose, cheeks and eyes of a person's face and comprises two apertures for the eyes of a person's face, such that the front side of the backing adhesively attaches to a skin surface of the person's face near the person's eyes; and
the second portion being contoured to the mouth and chin of a person's face and comprises an aperture corresponding to the mouth of a person's face, such that the front side of the backing adhesively attaches to a skin surface of the person's face near the person's mouth.

106. A method for improving the appearance of wrinkles in a mammal in need thereof, the method comprising applying to the surface of the skin of the mammal having the wrinkles or to the surface of the skin of the mammal at risk thereof, an adhesive patch of claim 1 for a period of time effective to improve the appearance of the wrinkles.

107. The method of claim 106 wherein the mammal is a human.

108. The method of claim 106 wherein the period of time is up to about 24 hours.

109. A method for exfoliating a skin surface of a mammal, the method comprising applying to the skin surface of the mammal in need of such exfoliation an adhesive patch of claim 1 for a period of time effective to adhere the adhesive skin patch to the skin surface, and removing the adhesive skin patch, thereby effectively exfoliating the skin surface.

110. The method of claim 109 wherein the mammal is a human.

111. The method of claim 109 wherein the period of time is up to about 24 hours.

112. A method for hydrating a skin surface of a mammal, the method comprising applying to the skin surface of the mammal in need of such hydration an adhesive patch of claim 1 for a period of time effective to hydrate the skin surface.

113. The method of claim 112 wherein the mammal is a human.

114. The method of claim 112 wherein the period of time is up to about 24 hours.

115. A method for firming a skin surface of a mammal, the method comprising applying to the skin surface of the mammal in need of such firming an adhesive patch of claim 1 for a period of time effective to hydrate the skin surface.

116. The method of claim 115 wherein the mammal is a human.

117. The method of claim 115 wherein the period of time is up to about 24 hours.
